(19) United States
(12) Patent Application Publication
(10) Pub. No.: US 2021/0349094 A1
(43) Pub. Date: Nov. 11, 2021

(54) DETECTION OF AUTOREACTIVE FECAL IMMUNOGLOBULIN A (IGA) FOR DIAGNOSIS OF LUPUS

(71) Applicant: MUSC Foundation for Research Development, Charleston, SC (US)

(72) Inventor: Chenthamarakshan Vasu, Charleston, SC (US)

(21) Appl. No.: 17/317,057

(22) Filed: May 11, 2021

Related U.S. Application Data
(60) Provisional application No. 63/022,809, filed on May 11, 2020.

(51) Int. Cl. G01N 33/577; A61K 45/06

(52) U.S. Cl. CPC G01N 33/577; G01N 2333/47; G01N 2800/50; A61K 45/06

(57) ABSTRACT

The invention provides a method for detecting an IgA anti-nuclear autoantibody in a fecal sample from a subject, wherein the presence of the IgA anti-nuclear autoantibody is an early indicator of the presence or increased risk of development of an autoimmune disease or disorder.

### CROSS REFERENCE TO RELATED APPLICATIONS

This application claims priority to U.S. Provisional Application No. 63/022,809, filed May 11, 2020 which is hereby incorporated by reference herein in its entirety.

### STATEMENT REGARDING FEDERALLY SPONSORED RESEARCH OR DEVELOPMENT

This invention was made with government support under R01AI138511 awarded by the National Institutes of Health. The government has certain rights in the invention.

### BACKGROUND OF THE INVENTION

Systemic lupus erythematosus (SLE) is a chronic autoimmune disorder that affects multiple organs including kidney, skin and nervous system (Lisnevskaia, et al., 2014, Lancet, 84:1878-1888; Dema and Charles, 2014, Discov Med, 17:247-255; Eroglu and Kohler, 2002, Ann Rheum Dis, 61:29-31; Chung et al., 2014, Lupus, 23:876-880). Women show higher SLE incidence rate (up to 9:1 ratio) than men (Grimaldi et al., 2005, Mol Immunol, 42:811-820; Ansar Ahmed et al., 1985, Am J Pathol 121:531-551; Bae et al., 1998, Arthritis and rheumatism, 41:2091-2099; Johnson et al., 1995, Arthritis and rheumatism, 38:551-558; Siegel et al., 1973, Seminars in arthritis and rheumatism, 3:1-54; Weckerle and Niewold, 2011, Clin Rev Allergy Immunol, 40:42-9). It is now believed that autoimmunity in SLE initiates several years before the clinical disease onset. However, an effective preventive therapy for SLE is still elusive due to a lack of reliable biomarkers that can predict the disease and inadequate understanding of the initiation and progression of autoimmunity in at-risk subjects.

Thus, there is a need in the art for non-invasive systems and methods for diagnosis as well as early detection of autoantibodies that are biomarkers of disease progression in autoimmune diseases including SLE. The present invention satisfies this need.

### SUMMARY OF THE INVENTION

In one embodiment, the invention relates to a method of detecting at least one IgA autoantibody in a subject comprising: obtaining a fecal sample from the subject; and contacting a portion of the sample with a capture molecule which specifically binds to at least one IgA autoantibody.

In one embodiment, the method detects total IgA, IgA1, IgA2 or IgA anti-nuclear antigen (IgA-ANA).

In one embodiment, the total IgA detected is total IgA1 or total IgA2.

In one embodiment, the IgA-ANA is an IgA1 or IgA2 subtype.

In one embodiment, the method further comprises contacting the sample with a secondary antibody, wherein the secondary antibody is linked to a detectable moiety; and measuring a detectable signal generated from the detectable moiety.

In one embodiment, at least one IgA anti-nuclear antigen autoantibody is an autoantibody against RNP/Sm, SS-A, Ro-52, SS-B, Scl-70, Pm-Scl, Jo-1, centromere B, PCNA, dsDNA, nucleosomes, nucleohistones, histones, ribozomal P-protein, or AMA-M2, or a natural or synthetic fragment thereof.

In one embodiment, the invention relates to a method of diagnosing a subject as having or being at increased risk of an autoimmune disease, the method comprising: a) obtaining a fecal sample from the subject; b) contacting a portion of the sample with a capture molecule which binds to at least one IgA autoantibody; c) contacting the sample with a detector molecule, wherein the detector molecule is linked to a detectable moiety; and d) examining a detectable signal generated from the detectable moiety visually or using an instrument. In one embodiment, steps b-d are achieved sequentially or simultaneously in an automated fashion.

In one embodiment, the capture molecule comprises an anti-IgA antibody which captures the total IgA antibodies, and the method further comprises a step of contacting the captured IgA total antibodies with at least one nuclear antigen prior to the step of contacting the sample with a detector molecule.

In one embodiment, the nuclear antigen is: RNP/Sm, SS-A, Ro-52, SS-B, Scl-70, Pm-Scl, Jo-1, centromere B, PCNA, dsDNA, nucleosomes, nucleohistones, histones, ribozomal P-protein, or AMA-M2, or a natural or synthetic fragment thereof.

In one embodiment, the capture molecule comprises a natural or synthetic nuclear antigen or a fragment thereof. In one embodiment, the nuclear antigen is: RNP/Sm, SS-A, Ro-52, SS-B, Scl-70, Pm-Scl, Jo-1, centromere B, PCNA, dsDNA, nucleosomes, nucleohistones, histones, ribozomal P-protein, or AMA-M2, or a fragment thereof.

In one embodiment, the capture molecule is an antibody against total IgA, IgA1 or IgA2 antibody, wherein the capture molecule is used to determine the abundance of IgA antibodies.

In one embodiment, the detector molecule is an antibody against IgA, IgA1 or IgA2 which is linked to a detectable moiety such as an enzyme, a colloidal or non-colloidal particle, a nanoparticle, a fluorescent material, or a luminescent material. In one embodiment, the detector molecule is an antibody against IgA, IgA1 or IgA2 which is linked to a detectable moiety such as HRP, colloidal gold or FITC.

In one embodiment, the method further comprises comparing the detectable signal generated from the detectable moiety to a comparator control, and diagnosing a subject as having or being at risk of developing an autoimmune disease when the detectable signal is higher relative to the comparator control.

In one embodiment, the comparator control is a negative control, an expected normal background value of the subject, a historical normal background value of the subject, an expected normal background value of a population that the subject is a member of, or a historical normal background value of a population that the subject is a member of.

In one embodiment, the disease or disorder is systemic lupus erythematosus (SLE), neonatal lupus erythematosus, Sjogren's Syndrome, Sicca syndrome, Diffuse systemic sclerosis, scleroderma, rheumatoid arthritis, Juvenile idiopathic arthritis, Drug induced lupus, Polymyositis, dermatomyositis, idiopathic inflammatory myopathies (IIM), mixed connective tissue disease (MCTD), or primary biliary cholangitis.

In one embodiment, the subject is asymptomatic or only exhibits non-specific indicators of the disease or disorder.

In one embodiment, the subject is receiving a cancer treatment therapeutic or biologic. In one embodiment, the subject is receiving Ipilimumab, Durvalumab, Avelumab, Atezolizumab, Pembrolizumab, Nivolumab, or Cemiplimab.

In one embodiment, the method further comprises administering a treatment for the diagnosed disease or disorder.

In one embodiment, the treatment is immunosuppressant drugs, corticosteroids, tofacitinib, calcineurin inhibitors, antiproliferative agents, mTOR inhibitors, abatacept, adalimumab, anakinra, certolizumab, etanercept, golimumab, infliximab, ixekizumab, natalizumab, secukinumab, tacilizumab, ustekinumab, vedolizumab, basiliximab, daclizumab, muromonab, hydroxychloroquine, methotrexate, cyclosporine, lifitegrast, nonsteroidal anti-inflammatory drugs, pilocarpine, or cevimeline.

In one embodiment, the invention relates to a method of preparing an IgA autoantibody sample comprising: providing a fecal sample comprising at least one IgA autoantibody, wherein the subject is suspected of having an autoimmune disease or disorder or the subject has a symptom of an autoimmune disease or disorder; selectively extracting total IgA antibodies from the fecal sample; and performing an assay on the extracted total IgA antibodies in order to quantitatively or qualitatively detect the level of IgA antibodies in the sample.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A and FIG. 1B depict the results from example experiments showing IgA+B cell frequency and IgA secretion in the intestinal mucosa. Single cell suspensions from Peyer's patch (PP) and small intestinal lamina propria (SiLP) were prepared from 16 week old SNF1 male, SNF1 female, and B6 female mice. FIG. 1A) These cells were subjected to FACS after staining with anti-mouse CD19 and IgA antibodies. Representative FACS plots (left) and mean±SD values (6 mice/group) of IgA+B cell frequencies (right) are shown. FIG. 1B) Single cell suspensions (5×10⁶ cells/ml) were cultured for 24 hours and supernatants were tested for spontaneously secreted total IgA levels by ELISA. Mean±SD of OD values (6 mice/group) are shown. P-value by Mann-Whitney test.

FIG. 2 depicts the results from example experiments showing the nuclear antigen reactivity of IgA produced by the gut mucosa cells. Immune cell rich fractions were prepared from single cell suspensions of small and large intestinal lamina propria (SiLP and LiLP) of 4 and 16 week old SNF1 males, SNF1 females and B6 females by Percoll gradient method. These cells were cultured (2×10⁶ cells/ml) for 48 hours in the presence of bacterial LPS and anti-CD40 antibody. Supernatants were tested for dsDNA and nucleohistone reactive IgA levels by ELISA. Mean±SD of OD values (5 mice/group) are shown. P-value by Mann-Whitney test.

FIG. 3A through FIG. 3C depict the results from example experiments demonstrating the abundance of IgA in the feces of lupus-prone and -resistant mice. Fecal samples were collected from individual SNF1 males, SNF1 females and B6 females at 4, 8, 12 and 16 weeks of age. FIG. 3A) Fecal pellets were subjected to ELISA to determine the IgA concentrations as detailed elsewhere herein. Mean±SD of OD values (16 mice/group) are shown for each time point. FIG. 3B) Fecal samples from an independent cohort of age-matched seropositive and seronegative mice (between 16 and 24 weeks of age) and sero and proteinuria-positive and seropositive and proteinuria negative (between 24 and 35 weeks of age) were also subjected to ELISA for IgA levels. Mean±SD of IgA concentration per gram feces (10 mice/group) are shown. FIG. 3C) Mice described for FIG. 3A were monitored for proteinuria levels for up to 40 weeks of age and the timing of severe proteinuria (protein: >5 mg/ml) onset was correlated with the 12- and 16-week fecal IgA levels of individual mice by Pearson's correlation approach.

FIG. 4A through FIG. 4C depict the results from example experiments demonstrating anti-dsDNA and nucleohistone reactive fecal IgA antibodies in SNF1 mice. Fecal samples were collected from individual SNF1 males, SNF1 females and B6 females at 8 and 16 weeks of age. FIG. 4A) Fecal pellets were subjected to ELISA to determine anti-dsDNA and nucleohistone reactivity as detailed in Materials and method. Mean±SD of ELISA titer per gram feces (16 mice/group) are shown for each time point. FIG. 4B) Fecal samples from an independent cohort of age-matched seropositive and seronegative mice (between 16 and 24 weeks of age) were also subjected to ELISA for anti-dsDNA and nucleohistone IgA levels. Mean±SD of ELISA titer per gram feces (10 mice/group) are shown. FIG. 4C) Mice described for FIG. 4A were monitored for proteinuria levels for up to 40 weeks of age and the timing of severe proteinuria (protein: >5 mg/ml) onset was correlated with the 16-week fecal anti-dsDNA and nucleohistone IgA levels of individual mice by Pearson's correlation approach.

FIG. 5A through FIG. 5E depict the results from example experiments demonstrating fecal IgA abundance and nAg reactivity in microbiota depleted mice. SNF1 females were left untreated or given broad spectrum antibiotic cocktail to deplete the gut microbiota. FIG. 5A) PP and LiLP cells from these mice at 16 weeks of age were subjected to FACS after staining using anti-mouse CD19 and IgA antibodies. Representative FACS plots (left) and mean±SD values (5 mice/group) of IgA+B cell frequencies (right) are shown. FIG. 5B & FIG. 5C) PP cells and immune cell rich fractions of LiLP were cultured (2×10⁶ cells/ml) for 48 hours in the presence of bacterial LPS and anti-CD40 antibody. Supernatants were tested for total IgA, dsDNA and nucleohistone reactive IgA levels by ELISA. Mean±SD of OD values (5 mice/group) are shown. FIG. 5D & FIG. 5E) Fecal pellets from another cohort of control and microbiota depleted mice (20 weeks of age) were subjected to ELISA to determine the total and dsDNA and nucleohistone reactive IgA levels. Mean±SD of OD values (8 mice/group) are shown. P-value by Mann-Whitney test.

FIG. 6 depicts the results from example experiments depicting IgA+B cell frequencies in lupus-susceptible and—non-susceptible mice. 8-wk-old SNF1 male and female and B6 female mice were euthanized, spleen, mesenteric lymph node (MLN) and Peyer's patch (PP) cells were stained and analyzed by FACS for IgA+B cell frequencies. Mean+/−SD values of 5 mice/group tested in triplicate are shown. P-values by non-parametric Mann-Whitney test.

FIG. 7 depicts the results from example experiments demonstrating IgA antibody production by GALT cells from lupus-susceptible and—non-susceptible mice and the nAg reactivity of these IgA antibodies. 8-wk-old SNF1 male and female and B6 female mice were euthanized, equal number (2.5×10⁶ cells/ml) of Peyer's patch (PP) cells and percoll-centrifugation enriched small intestinal lamina propria (SiLP) immune cells were cultured for 48 h with Bacterial LPS and anti-CD40 antibody. Supernatants were tested for total IgA concentration and dsDNA reactive IgA antibody levels by ELISA as previoulsy described. IgA concentrations were determined using a standard curve generated using purified mouse IgA. Blank/background subtracted OD values are shown for dsDNA reactive IgA. Mean+/−SD values of 6 mice/group tested in triplicate are shown. P-values by non-parametric Mann-Whitney test.

FIG. 8 depicts the results from example experiments demonstrating the fecal IgA—abundance and -nAg reactivity of seronegative age lupus-susceptible and—non-susceptible mice. Fecal pellets collected from 12-wk-old SNF1-male and -female and B6-female mice, PBS soluble extracts were prepared from pool of 3-4 pellets of each mouse processed separately, and subjected to ELISA to determine total IgA concentration as well as nAg (dsDNA and nucleohistone, NH) -reactive IgA levels. IgA concentrations were determined for optimally diluted initial extract using a standard curve generated using purified mouse IgA. nAg reactivity was determined by testing multiple dilutions (starting at 1:5 dilution) of extracts. Highest dilution that showed OD value >0.05 above the background was considered initial titer of the extract. Initial extract was made by overnight homogenization of 50 mg pellet/ml PBS. Final-concentration and -titer values were calculated for per gram feces and shown. Mean+/−SD values of 10 mice/group tested in duplicate are shown. P-values by non-parametric Mann-Whitney test.

FIG. 9 depicts the results from example experiments demonstrating the fecal IgA-abundance and -nAg reactivity of sero- and proteinuria-positive lupus-susceptible mice and age-matched non-susceptible mice. Fecal pellets collected from proteinuria and serum anti-dsDNA IgG positive SNF 1-female and B6-female mice (both: >24 weeks of age) were subjected to ELISA to determine total IgA and nAg (dsDNA)-reactive IgA titers (/gram feces) were calculated. nAg reactivity index (NRI) for each sample was calculated by dividing nAg reactive IgA titer with respective IgA concentration. Mean+/−SD values of 5 mice/group tested in duplicate are shown. P-values by non-parametric Mann-Whitney test. All values were calculated for per gram feces.

FIG. 10 depicts the results from example experiments demonstrating fecal total IgA and IgA-isotype abundances in healthy controls (HC) and SLE patients. Cryopreserved surplus stool samples of 12 HCs and 12 SLE patients were subjected to ELISA to determine human IgA, IgA1 and IgA2 concentrations using anti-IgA, -IgA1 and -IgA2 monoclonal antibody coated plates and purified human IgA, IgA1 and IgA2 as standards. All values were calculated for per gram dry feces. Top panel; heatmap showing relative concentrations of IgA, IgA1 and IgA2 in each sample side-by-side. Lower panel: mean+/−SD values of 12 subjects/group tested in duplicate. P-values by non-parametric Mann-Whitney test.

FIG. 11A and FIG. 11B depict the results from example experiments demonstrating nAg reactivity of fecal total-IgA of healthy controls (HC) and SLE patients. FIG. 11A) Stool sample aliquots described under FIG. 5 were subjected to ELISA to determine nAg (dsDNA and nucleohistone, NH) reactive IgA levels using dsDNA and NH coated plates. All values were calculated for per gram dry feces. nAg reactivity index (NRI) for each sample was calculated by dividing NH and dsDNA reactive IgA titers with IgA concentration of respective sample as described for FIG. 4. mean+/−SD values of 12 subjects/group tested in duplicate are shown. P-values by non-parametric Mann-Whitney test. FIG. 11B) Representative HC and SLE group stool extracts (2 each) were used in ANA assay to detect nAg reactivity of fecal IgA. Hep2 cell substrate slide and Alexa Fluor488 linked anti-human IgA were used.

FIG. 12 depicts results from example experiments demonstrating the abundance and nAg reactivity of fecal total-IgA of healthy controls (HC) and first degree relatives of SLE patients with no clinical disease. Stool extracts were subjected to ELISA to determine total IgA concentration and nAg (dsDNA and nucleohistone, NH) reactive IgA levels using dsDNA and NH coated plates. mean+/−SD values of 10 subjects/group tested in duplicate are shown. P-values by t-test. IgA concentration (left panel) and nAg titers (middle and right panels) were calculated for per gram dry feces.

FIG. 13 depicts results from example experiments demonstrating nAg reactivity of fecal IgA1 and IgA2 subtypes of healthy controls (HC) and SLE patients. Stool sample extracts were subjected to ELISA to determine nAg (dsDNA and nucleohistone, NH) reactive IgA1 and IgA2 subtype levels using dsDNA and NH coated plates. All titer values were calculated for per gram feces. mean+/−SD values of a total of 5 subjects/group tested in duplicate are shown. P-values by non-parametric Mann-Whitney test.

FIG. 14 depicts results from example experiments demonstrating the abundance and nuclear antigen reactivity of fecal IgA antibodies in lupus-prone MRL/Lpr mice at pre-clinical stage. Fecal samples were collected from individual lupus-prone MRL/lpr males (n=14) and MRL/lpr females (n=13), and lupus-resistant B6 females (n=8) at 8 weeks of age. Fecal pellets were subjected to ELISA to determine the concentration, or anti-dsDNA and nucleohistone reactivity, of IgA. Mean±SD of concentration (mg)/gram feces (left panel) or ELISA titer per gram feces (middle and right panels) are shown. P-values by non-parametric Mann-Whitney test.

FIG. 15 depicts results from example experiments demonstrating the abundance and nuclear antigen reactivity of fecal IgA antibodies in lupus-prone NZBW-F1 mice at pre-clinical stage. Fecal samples were collected from individual lupus-prone NZBW-F1 males (n=14) and NZBW-F1 females (n=11), and lupus-resistant B6 females (n=8) at 8 weeks of age. Fecal pellets were subjected to ELISA to determine the concentration, or anti-dsDNA and nucleohistone reactivity, of IgA. Mean±SD of concentration (mg)/gram feces (left panel) or ELISA titer per gram feces (middle and right panels) are shown. P-values by non-parametric Mann-Whitney test.

FIG. 16 depicts results from example experiments demonstrating the abundance and nuclear antigen reactivity of fecal IgA antibodies in lupus-prone NZM2410 mice at pre-clinical stage. Fecal samples were collected from individual lupus-prone NZM2410 males (n=8) and NZM2410 females (n=8), and lupus-resistant B6 females (n=8) at 8 weeks of age. Fecal pellets were subjected to ELISA to determine the concentration, or anti-dsDNA and nucleohistone reactivity, of IgA. Mean±SD of concentration (mg)/gram feces (left panel) or ELISA titer per gram feces (middle and right panels) are shown. P-values by non-parametric Mann-Whitney test. These results show higher abundance and nuclear antigen reactivity of fecal IgA in both male and female lupus-prone NZM2140 mice compared to non-susceptible B6 mice. Unlike in SNF1 mice which showed gender bias, NZM2140 mice did not show significant gender bias in fecal IgA features.

### DETAILED DESCRIPTION

The present invention relates to assay systems and methods for detecting one or more of total IgA, IgA subtype abundance and IgA-anti-nuclear antigen (IgA-ANA) autoantibodies in a fecal sample of a subject in need thereof. Total IgA, as used herein, includes total IgA1 or total IgA2 subtypes.

In one embodiment, the IgA-ANA is an autoantibody against RNP/Sm, SS-A, Ro-52, SS-B, Scl-70, Pm-Scl, Jo-1, centromere B, PCNA, dsDNA, nucleosomes, histones, ribozomal P-protein, or AMA-M2.

In one embodiment, the invention relates to methods to diagnose the presence or an increased risk of development of an autoimmune disease or disorder, such as lupus. In one embodiment, the invention relates to methods of treating a subject identified as having or being at increased risk of developing an autoimmune disease or disorder. In one embodiment, the disease or disorder is associated with higher antibody production in the gut and excreted in feces. In one embodiment, the antibody is IgA. In one embodiment, the disease or disorder is associated with an IgA-anti-nuclear antigen (IgA-ANA) autoantibody.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein the terms “alteration,” “defect,” “variation,” or “mutation,” refers to a mutation in a gene in a cell that affects the function, activity, expression (transcription or translation) or conformation of the polypeptide that it encodes. Mutations encompassed by the present invention can be any mutation of a gene in a cell that results in the enhancement or disruption of the function, activity, expression or conformation of the encoded polypeptide, including the complete absence of expression of the encoded protein and can include, for example, missense and nonsense mutations, insertions, deletions, frameshifts and premature terminations. Without being so limited, mutations encompassed by the present invention may alter splicing the mRNA (splice site mutation) or cause a shift in the reading frame (frameshift).

The term “amplification” refers to the operation by which the number of copies of a target nucleotide sequence present in a sample is multiplied.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “marker” or “biomarker” is meant to include a parameter which is useful according to this invention for determining the presence and/or severity of a disease or disorder.

The level of a marker or biomarker “significantly” differs from the level of the marker or biomarker in a reference sample if the level of the marker in a sample from the patient differs from the level in a sample from the reference subject by an amount greater than the standard error of the assay employed to assess the marker, and preferably at least 10%, and more preferably 25%, 50%, 75%, or 100%.

The term “control or reference standard” describes a material comprising none, or a normal, low, or high level of one of more of the marker (or biomarker) expression products of one or more the markers (or biomarkers) of the invention, such that the control or reference standard may serve as a comparator against which a sample can be compared.

By the phrase “determining the level of marker (or biomarker) expression” is meant an assessment of the degree of expression of a marker in a sample at the nucleic acid or protein level, using technology available to the skilled artisan to detect a sufficient portion of any marker expression product.

“Differentially increased expression” or “up regulation” refers to biomarker product levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween than a control.

“Differentially decreased expression” or “down regulation” refers to biomarker product levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments therebetween than a control.

A “disease” is a state of health of an animal wherein the animal cannot maintain homeostasis, and wherein if the disease is not ameliorated then the animal's health continues to deteriorate.

As used herein, an “instructional material” includes a publication, a recording, a diagram, or any other medium of expression which can be used to communicate the usefulness of a component of the invention in a kit for detecting biomarkers disclosed herein. The instructional material of the kit of the invention can, for example, be affixed to a container which contains the component of the invention or be shipped together with a container which contains the component. Alternatively, the instructional material can be shipped separately from the container with the intention that the instructional material and the component be used cooperatively by the recipient.

The term “label” when used herein refers to a detectable compound or composition that is conjugated directly or indirectly to a probe to generate a “labeled” probe. The label may be detectable by itself with or without chemical reaction (e.g. radioisotope labels, colloidal gold or colored latex or polystyrene particles, or fluorescent labels) or, in the case of an enzymatic label, may catalyze chemical alteration of a substrate compound or composition that is detectable (e.g., avidin-biotin). In some instances, primers can be labeled to detect a PCR product.

The “level” of one or more biomarkers means the absolute or relative amount or concentration of the biomarker in the sample.

The term “marker (or biomarker) expression” as used herein, encompasses the transcription, translation, post-translation modification, and phenotypic manifestation of a gene, including all aspects of the transformation of information encoded in a gene into RNA or protein. By way of non-limiting example, marker expression includes transcription into messenger RNA (mRNA) and translation into protein, as well as transcription into types of RNA such as transfer RNA (tRNA) and ribosomal RNA (rRNA) that are not translated into protein.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “providing a prognosis” refers to providing a prediction of the probable course and outcome of a disease or disorder, including prediction of severity, duration, chances of recovery, etc. The methods can also be used to devise a suitable therapeutic plan.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype.

“Sample” or “biological sample” as used herein means a biological material isolated from an individual. The biological sample may contain any biological material suitable for detecting the desired biomarkers, and may comprise cellular and/or non-cellular material obtained from the individual.

“Standard control value” as used herein refers to a predetermined amount of a particular protein or nucleic acid that is detectable in a sample, such as a fecal sample, either in whole feces or in fecal supernatant. The standard control value is suitable for the use of a method of the present invention, in order for comparing the amount of a protein or nucleic acid of interest that is present in a fecal sample. An established sample serving as a standard control provides an average amount of the protein or nucleic acid of interest in the feces that is typical for an average, healthy person of reasonably matched background, e.g., gender, age, ethnicity, and medical history. A standard control value may vary depending on the protein or nucleic acid of interest and the nature of the sample (e.g., whole feces or fecal supernatant).

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### Description

The present invention relates to methods for autoantibody detection. In some aspects, autoantibodies are detected from feces of patients using the developed assay.

In some embodiments, the methods relate to the detection of IgA, including but not limited to total IgA, IgA1 and IgA2. In some embodiments, the methods relate to the detection of anti-nuclear antigen IgA antibodies (IgA-ANA) in a fecal sample. In various embodiments, the IgA-ANA is IgA, IgA1 or IgA2. Non-limiting examples of such detectible IgA-ANA include autoantibodies against RNP/Sm, SS-A, Ro-52, SS-B, Scl-70, Pm-Scl, Jo-1, centromere B, PCNA, dsDNA, nucleosomes, nucleohistone, histones, ribozomal P-protein, and AMA-M2. In some embodiments, the IgA-ANA can be used to detect natural or synthetic antigens, full-length antigens, antigenic fragments, peptides or oligonucleotides (e.g., shredded nucleic acids). It should be appreciated that any number of IgA-ANA can be detected according to the methods of the invention, including, without limitation, 1, 2, 3, 4, 5, 6, 7, 8, 9, 10 or more than 10 IgA-ANA. In some embodiments, the methods relate to the detection of the abundance of IgA antibodies (IgA, IgA1 or IgA2) in a fecal sample along with IgA-ANA detection.

The noninvasive early detection of at least one of total IgA and IgA-ANA in a subject via the present invention enables clinicians to identify the presence of an autoimmune disease or disorder at pre-clinical or clinical stage in a fast, economical and non-invasive manner.

### Methods of Detecting Autoantibodies

In some embodiments, the present invention relates to methods of detecting the levels of total IgA antibodies in a fecal sample of a subject. In some embodiments, the present invention relates to methods of detecting the levels IgA1 subtype antibodies in a fecal sample of a subject. In some embodiments, the present invention relates to methods of detecting the levels IgA2 subtype antibodies in a fecal sample of a subject. In some embodiments, the present invention relates to methods of detecting at least one IgA-ANA autoantibody in a fecal sample of a subject. Immunochemical techniques for detecting antibodies against specific antigens are well known in the art, as are techniques for detecting specific antigens themselves. Detection of an antibody will typically involve contacting a sample with a capture antigen, wherein a binding reaction between the sample and the capture antigen indicates the presence of the antibody of interest.

In some embodiments, the method includes isolating total IgA, IgA1 or IgA2 from a fecal sample, and then testing the isolated IgA antibodies for anti-ANA activity. Methods of measuring total IgA, IgA1 and IgA2 antibodies include contacting the sample with capture molecules against IgA, IgA1 and IgA2 respectively. Methods of measuring anti-ANA activity are well known in the art and include, but are not limited to, contacting the total IgA with an antigen recognized by an anti-ANA antibody.

A capture antigen for a biomarker antibody can be a natural antigen recognized by the auto-antibody (e.g. RNP/Sm, SS-A, Ro-52, SS-B, Scl-70, Pm-Scl, Jo-1, centromere B, PCNA, dsDNA, nucleosomes, nucleohistone, histones, ribozomal P-protein, and AMA-M2 or a fragment thereof), or it may be an antigen comprising an epitope of the natural antigen which is recognized by the auto-antibody. It may be an isolated natural protein, a recombinant protein or a synthetic protein or peptide. It may be an isolated natural nucleic acid molecule, a recombinant nucleic acid molecule or synthetic nucleic acid molecule. Where a detection antigen is a polypeptide its amino acid sequence can vary from the natural sequences disclosed above, provided that it has the ability to specifically bind to an auto-antibody of the invention (i.e. the binding is not non-specific and so the detection antigen will not arbitrarily bind to antibodies in a sample). It may even have little in common with the natural sequence (e.g. a mimotope, an aptamer, etc.). Typically, though, a capture antigen will comprise (i) an amino acid sequence having at least 90% (e.g. 91%, 92%, 93%, 94%, 95%, 96%, 97%, 98%, 99%) sequence identity to the native antigenic polypeptide sequence, or (ii) comprise a structure that mimics the structure of a naturally occurring dsDNA molecule. Thus the detection antigen may be one of the variants discussed above.

Capture antigens can be purified from human or non-human sources, or can be synthetic or recombinant antigens (particularly where the capture antigen uses sequences which are not present in the natural antigen e.g. for attachment). Synthetic capture antigens include, but are not limited to synthetic polypeptides and synthetic oligonucleotides (e.g., synthetic DNA or RNA.) Various systems are available for recombinant expression, and the choice of system may depend on the auto-antibody to be detected. For example, prokaryotic expression (e.g. using E. coli) is useful for detecting many auto-antibodies, but eukaryotic expression may be required for some capture antigens. For example, if an auto-antibody recognizes a specific discontinuous epitope then a recombinant expression system which provides correct protein folding may be required.

Various assay formats can be used for detecting autoantibodies in samples. For example, the invention may use one or more of immunochromatography, western blot, immunoprecipitation, silver staining, mass spectrometry (e.g. MALDI-MS), conductivity-based methods, dot blot, slot blot, dip stick, colorimetric methods, fluorescence-based detection methods, or any form of immunoassay, etc. The binding of antibodies to antigens can be detected by any means, including enzyme-linked assays such as ELISA, immunochromatographic assays, immunodot assays, suspension bead assay, flow-cytometry based assay, radioimmunoassays (RIA), immunoradiometric assays (IRMA), immunoenzymatic assays (IEMA), DELFIA™ assays, surface plasmon resonance or other evanescent light techniques (e.g. using planar waveguide technology), label-free electrochemical sensors, and indirect assays for immunological methods.

The capture antigen may be a fusion polypeptide with a first region and a second region, wherein the first region can react with an auto-antibody in a sample and the second region can react with a substrate or solid surface to immobilize the fusion polypeptide thereon.

In some embodiments, the substrate or solid surface may comprise a strip, a slide, a filter paper, a nitrocellulose, PVDF or nylon membrane, a bead, a microsphere or nanoparticle, a well of a microtitre plate, a conductive surface suitable for performing mass spectrometry analysis, a semiconductive surface, a surface plasmon resonance support, a planar waveguide technology support, a microfluidic devices, or any other device or technology suitable for detection of antibody-antigen binding.

In one embodiment, the method may be performed as an immunoassay assay and includes the steps of obtaining a sample from the subject, contacting the sample with a capture molecule which specifically binds an autoantibody to be detected, or a fragment thereof.

Next, in some embodiments, the bound antibodies are detected. In certain embodiments, a detector molecule is used, where the detector molecule binds to the autoantibody-capture molecule complex. In one embodiment, a secondary antibody that binds to the autoantibody to be detected is used as a direct or indirect detector molecule.

In one embodiment, the method relates to methods of detecting total IgA. In some embodiments, detection of IgA auto-antibodies is also possible e.g. by using a detection reagent which recognizes the appropriate class of auto-antibody (total IgA, IgA1 or IgA2). In some embodiments, the detector molecule of the invention is able to distinguish between different antibody subtypes and/or isotypes. In some embodiments, the detector molecule of the invention is specific for IgA antibody isotypes.

The detector molecules (e.g, secondary antibodies) can be labeled, such as with a fluorescent molecule, fluorescein isothiocyanate, Alexa Fluor, colloidal gold, colored latex particles, HRP, alkaline phosphatase, Biotin, or any other label known in the art. The detectable moiety may be measured, quantitatively or qualitatively, with or without specialized instruments. The detectable moiety may be measured to determine the presence or absence of at least one autoantibody in the sample.

In one embodiment, the secondary antibody is labeled with a detectable moiety which generates a fluorescent signal which can be detected. In one embodiment, the secondary antibody is labeled with biotin and is then contacted with a streptavidin bound molecule for generating a detectable readout, allowing for indirect detection of the secondary antibody bound to the autoantibody:capture molecule complex. In one embodiment, the detectable moiety generates a current which can be detected. In one embodiment, the secondary antibody is labeled with colored substance such as colloidal gold or microspheres/particles. In one embodiment, the streptavidin bound molecule comprises poly-horseradish peroxidase. For example, in one embodiment horseradish peroxidase in casein-phosphate-buffered saline can be used, and a 3,3′,5,5′-tetramethylbenzidine substrate for horseradish peroxidase can be provided, allowing for generation of an amperometric signal which can be measured.

In one embodiment, the present invention provides methods for diagnosing, determining risk or treating a disease or disorder associated with at least one autoantibody in a subject. Accordingly, the present invention features methods for identifying subjects who are at risk of developing, or having developed, one or a combination of autoimmune diseases including, but not limited to, systemic lupus erythematosus (SLE), neonatal lupus erythematosus, Sjogren's Syndrome, Sicca syndrome, Diffuse systemic sclerosis, scleroderma, rheumatoid arthritis, Juvenile idiopathic arthritis, Drug induced lupus, Polymyositis, dermatomyositis, idiopathic inflammatory myopathies (IIM), mixed connective tissue disease (MCTD), and primary biliary cholangitis (PBC; previously referred to as primary biliary cirrhosis), including those subjects who are asymptomatic or only exhibit non-specific indicators of the disease or disorder.

In a number of specific autoimmune diseases, such as SLE, elevated IgA, IgA1 and IgA2, and IgA-ANA autoantibodies appear before the disease clinical onset is presented, and IgA-ANA can be detected in a fecal sample prior to detection in other biological samples, including but not limited to, serum. Therefore, in some embodiments, the invention provides methods for early detection and diagnosis of autoimmune diseases. In one embodiment, the invention provides methods for early detection and diagnosis of SLE.

The invention is used for early detection and diagnosis of the disease in a subject at any age. In some embodiments the subject is female and at least 10 years old (e.g. >10, >15, >20, >25, >30, >35, >40, >45, >50, >55, >60, >65, >70). In some embodiments the subject is at least of child-bearing age as the risk of lupus increases in this age group. In some embodiments the subject is a post-menopausal female.

The subject may be pre-symptomatic for SLE or may already be displaying clinical symptoms. For pre-symptomatic subjects the invention is useful for predicting that symptoms may develop in the future if no preventative action is taken. For subjects already displaying clinical symptoms, the invention may be used to confirm or resolve another diagnosis. The subject may already have begun treatment for lupus.

In some embodiments the subject may already be known to be predisposed to development of SLE e.g. due to family or genetic links. In other embodiments, the subject may have no such predisposition, and may develop the disease as a result of environmental factors e.g. as a result of exposure to particular chemicals (such as toxins or pharmaceuticals), as a result of diet, of infection, of oral contraceptive use, of postmenopausal use of hormones, etc.

Because the invention can be implemented relative easily and cheaply it is not restricted to being used in patients who are already suspected of having SLE. Rather, it can be used to screen the general population or a high risk population.

In some embodiments, the invention provides methods for detection and diagnosis of SLE or an increased risk of developing SLE in juvenile subjects who are asymptomatic or only exhibit non-specific indicators of the disease or disorder. In some embodiments, juvenile human subjects are 20 years old or less. In some embodiments, juvenile human subjects are 19 years old, 18 years old, 17 years old, 16 years old, 15 years old, 14 years old, 13 years old, 12 years old, 11 years old, 10 years old, or less than 10 years old.

The methods of the invention are also useful for monitoring subjects undergoing treatments and therapies for an autoimmune disease or disorder associated with at least one autoantibody, and for selecting or modifying therapies and treatments that would be efficacious in subjects having an autoimmune disease or disorder, wherein selection and use of such treatments and therapies slow the progression of one or more autoimmune disease, or prevent their onset.

The invention provides improved diagnosis and prognosis of SLE or another autoimmune disease or disorder associated with at least one autoantibody. The risk of developing an autoimmune disease or disorder associated with at least one autoantibody can be assessed by measuring one or more autoantibody as described herein, and comparing the measured values to reference or index values. Subjects identified as having an increased level of at least one of an autoantibody can optionally be selected to receive treatment regimens, such as administration of prophylactic or therapeutic compounds or treatments to prevent, treat or delay the onset of an autoimmune disease or disorder associated with at least one autoantibody.

Identifying a subject before they develop an autoimmune disease or disorder associated with at least one autoantibody enables the selection and initiation of various therapeutic interventions or treatment regimens in order to delay, reduce or prevent the development or severity of the disease or disorder. In certain instances, monitoring the levels of at least one autoantibody also allows for the course of treatment of the disease or disorder to be monitored. For example, a sample can be provided from a subject undergoing treatment regimens or therapeutic interventions (e.g., drug treatments, immunosuppressive therapy, etc.) for an autoimmune disease or disorder. Samples can be obtained from the subject at various time points before, during, or after treatment.

Many cancer therapy biologicals and other drugs induce autoimmunity including lupus like conditions. This invention may be useful for early detection of autoimmunity induced by drugs such as cancer therapy check-point drugs and biologicals. The present invention also provides a method of screening for susceptibility to drug induced autoimmunity at various stages of cancer therapy. Cancer therapeutic agents associated with an increased risk of autoimmunity include, but are not limited to Ipilimumab, Durvalumab, Avelumab, Atezolizumab, Pembrolizumab, Nivolumab, and Cemiplimab. Therefore, in one embodiment, the methods of the invention can be used to monitoring the levels of at least one autoantibody in subjects undergoing treatment for cancer.

Data concerning the presence or levels of the autoantibodies of the present invention can also be combined or correlated with other data or test results, including but not limited to imaging data, medical history and any relevant family history.

The present invention also provides methods for identifying agents for treating an autoimmune disease or disorder that are appropriate or otherwise customized for a specific subject. In this regard, a test sample from a subject, exposed to a therapeutic agent, drug, or other treatment regimen, can be taken and the level of one or more autoantibody can be determined. The level of the autoantibody can be compared to a sample derived from the subject before and after treatment, or can be compared to samples derived from one or more subjects who have shown improvements in risk factors as a result of such treatment or exposure.

In some embodiments, these methods may utilize a fecal sample for the detection of one or more autoantibody in the sample. In one embodiment, the sample is a fecal supernatant sample. Frequently the sample will be a “clinical sample” which is a sample derived from a patient.

In various embodiments, the level of one or more of markers of the invention in the biological sample of the subject is compared with the level of a corresponding biomarker in a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of.

In some embodiments, the invention provides methods of diagnosing, monitoring the progression of, or treating an autoimmune disease or disorder associated with at least one of an IgA-ANA in a subject by assessing the level of one or more of an IgA-ANA in a biological sample of the subject.

Information obtained from the methods of the invention described herein can be used alone, or in combination with other information (e.g., disease status, disease history, vital signs, blood chemistry, etc.) from the subject or from the biological sample obtained from the subject.

In some embodiments, the level of one or more IgA-ANA is determined to be increased when the level of the IgA-ANA detected in a biological sample of a subject is increased by at least 10%, by at least 20%, by at least 30%, by at least 40%, by at least 50%, by at least 60%, by at least 70%, by at least 80%, by at least 90%, or by at least 100%, when compared to with a comparator control.

In one embodiment, a fecal sample from a subject is assessed for the level of one or more IgA-ANA autoantibody. In some embodiments, the level of one or more IgA-ANA autoantibody of the invention is determined to be increased when the level of one or more IgA-ANA autoantibody detected in a biological sample of a subject is increased by at least 10%, by at least 20%, by at least 30%, by at least 40%, by at least 50%, by at least 60%, by at least 70%, by at least 80%, by at least 90%, or by at least 100%, when compared to with a comparator control.

In some embodiments, the invention involves a step of determining the level of one or more IgA-ANA autoantibody. In some embodiments of the invention this determination can be a simple yes/no determination, whereas other embodiments may require a quantitative or semi-quantitative determination, still other embodiments may involve a relative determination (e.g. a ratio relative to another marker, or a measurement relative to the same marker in a control sample), and other embodiments may involve a threshold determination (e.g. a yes/no determination whether a level is above or below a threshold). Usually biomarkers will be measured to provide quantitative or semi-quantitative results (whether as relative concentration, absolute concentration, titre, etc.) as this gives more data for use with classifier algorithms.

In some embodiments, the raw data obtained from an assay for determining the presence, absence, or level (absolute or relative) require some sort of manipulation prior to their use. For instance, the nature of most detection techniques means that some signal will sometimes be seen even if no antibody is actually present and so this noise may be removed before the results are interpreted. Similarly, there may be a background level of the antibody in the general population which needs to be compensated for. Data may need scaling or standardizing to facilitate inter-experiments comparisons. These and similar issues, and techniques for dealing with them, are well known in the immunodiagnostic area.

Various techniques are available to compensate for background signal in a particular experiment. For example, replicate measurements will usually be performed to determine intra-assay variation, and average values from the replicates can be compared. Furthermore, standard markers can be used to determine inter-assay variation and to permit calibration and/or normalization e.g. an array can include one or more standards for indicating whether measured signals should be proportionally increased or decreased. For example, an assay might include a step of analyzing the level of one or more control marker(s) in a sample e.g. levels of an antigen or antibody unrelated to SLE.

The level of a biomarker relative to a single baseline level may be defined as a fold difference. Normally it is desirable to use techniques that can indicate a change of at least 1.5-fold e.g. 1.75-fold, 5-fold, etc.

As well as compensating for variation which is inherent between different experiments, it can also be important to compensate for background levels of a biomarker which are present in the general population. Again, suitable techniques are well known. For example, levels of a particular auto-antibody in a sample will usually be measured quantitatively or semi-quantitatively to permit comparison to the background level of that biomarker. Various controls can be used to provide a suitable baseline for comparison, and choosing suitable controls is routine in the diagnostic field. Further details of suitable controls are given below.

The measured level(s) of at least one IgA-ANA after any compensation/normalization/etc., can be transformed into a diagnostic result in various ways. This transformation may involve an algorithm which provides a diagnostic result as a function of the measured level(s).

The creation of algorithms for converting measured levels or raw data into scores or results is well known in the art. For example, linear or non-linear classifier algorithms can be used. These algorithms can be trained using data from any particular technique for measuring the marker(s). Suitable training data will have been obtained by measuring the biomarkers in “case” and “control” samples i.e. samples from subjects known to suffer from SLE and from subjects known not to suffer from SLE. Most usefully the control samples will also include samples from subjects with a related disease which is to be distinguished from the disease of interest e.g. it is useful to train the algorithm with data from rheumatoid arthritis subjects and/or with data from subjects with connective tissue diseases other than lupus. The classifier algorithm is modified until it can distinguish between the case and control samples e.g. by adding or removing markers from the analysis, by changes in weighting, etc. Thus a method of the invention may include a step of analyzing biomarker levels in a subject's sample by using a classifier algorithm which distinguishes between SLE subjects and non-SLE subjects based on measured biomarker levels in samples taken from such subjects. Various suitable classifier algorithms are available e.g. linear discriminant analysis, naïve Bayes classifiers, perceptrons, support vector machines (SVM) and genetic programming (GP).

In some embodiments, the methods of the invention involve determining whether a fecal sample contains an IgA-ANA level which is associated with lupus. Thus a method of the invention can include a step of comparing IgA-ANA levels in a test subject's sample to levels in (i) a sample from a patient with SLE and/or (ii) a sample from a subject without SLE. The comparison provides a diagnostic indicator of whether the test subject has SLE. An increased level of IgA-ANA indicates that the test subject has SLE.

The level of an IgA-ANA in a fecal sample should be significantly different from that seen in a negative control. Advanced statistical tools can be used to determine whether two levels are the same or different. For example, an in vitro diagnosis will rarely be based on comparing a single determination. Rather, an appropriate number of determinations will be made with an appropriate level of accuracy to give a desired statistical certainty with an acceptable sensitivity and/or specificity. Antibody levels can be measured quantitatively to permit proper comparison, and enough determinations will be made to ensure that any difference in levels can be assigned a statistical significance to a level of p<0.05 or better. The number of determinations will vary according to various criteria (e.g. the degree of variation in the baseline, the degree of up-regulation in disease states, the degree of noise, etc.) but, again, this falls within the normal design capabilities of a person of ordinary skill in this field. For example, interquartile differences of normalized data can be assessed, and the threshold for a positive signal (i.e. indicating the presence of a particular auto-antibody) can be defined as requiring that antibodies in a sample react with a diagnostic antigen at least 2.5-fold more strongly that the interquartile difference above the 75th percentile. Other criteria are familiar to those skilled in the art and, depending on the assays being used, they may be more appropriate than quantile normalization. Other methods to normalize data include data transformation strategies known in the art e.g. scaling, log normalization, median normalization, etc.

Methods of the invention may have sensitivity of at least 70% (e.g. >70%, >75%, >80%, >85%, >90%, >95%, >96%, >97%, >98%, >99%). Methods of the invention may have specificity of at least 70% (e.g. >70%, >75%, >80%, >85%, >90%, >95%, >96%, >97%, >98%, >99%). In some embodiments, methods of the invention may have both specificity and sensitivity of at least 70% (e.g. >70%, >75%, >80%, >85%, >90%, >95%, >96%, >97%, >98%, >99%).

Data obtained from methods of the invention, and/or diagnostic information based on those data, may be stored in a computer medium (e.g. in RAM, in non-volatile computer memory, on CD-ROM) and/or may be transmitted between computers e.g. over the internet.

If a method of the invention indicates that a subject has, or is at increased risk of developing SLE, further steps may then follow. For instance, the subject may undergo confirmatory diagnostic procedures, such as those involving physical inspection of the subject, and/or may be treated with therapeutic agent(s) suitable for treating SLE.

### Methods of Treatment

The present invention provides methods of testing or determining the need for, as well as efficacy of, treatment regiments for an autoimmune disease or disorder. The present invention also provides a method of diagnosing, treating or preventing an autoimmune disease or disorder, or reducing at least one symptom associated with an autoimmune disease or disorder in a subject. In one embodiment, the present invention provides a method of preparing a diagnostic assay for assessing the need for treating or preventing an autoimmune disease or disorder, or reducing at least one symptom associated with an autoimmune disease or disorder in a subject. In one embodiment, the present invention provides a method of performing a diagnostic assay for assessing the need for treating or preventing an autoimmune disease or disorder, or reducing at least one symptom associated with an autoimmune disease or disorder in a subject. In one embodiment, the method comprises administering an effective amount of a therapeutic composition to, or performing a therapeutic procedure on, a subject identified by the methods of the invention as having or being at increased risk of developing an autoimmune disease or disorder through detection of an autoantibody in a biological sample of the subject.

In one embodiment, the therapeutic composition comprises at least one therapeutic agent to treat the patient's disease or disorder. In one embodiment, the therapeutic composition comprises at least one therapeutic agent to reducing at least one symptom associated with the patient's disease or disorder.

Exemplary therapeutic agents that can be administered to subjects identified as having or at increased risk of developing an autoimmune disease or disorder according to the methods of the invention include, but are not limited to, immunosuppressant drugs including, but not limited to, corticosteroids (e.g., prednisone, budesonide, and prednisolone), tofacitinib, calcineurin inhibitors (e.g., tacrolimus and cyclosporine), antiproliferative agents (e.g., mycophenolate mofetil, mycophenolate sodium, leflunomide and azathioprine), mTOR inhibitors (e.g., sirolimus and everolimus), biologics (e.g., abatacept, adalimumab, anakinra, certolizumab, etanercept, golimumab, infliximab, ixekizumab, natalizumab, secukinumab, tacilizumab, ustekinumab, and vedolizumab) and monoclonal antibodies (e.g., basiliximab, daclizumab, and muromonab), hydroxychloroquine, methotrexate, cyclosporine, lifitegrast, nonsteroidal anti-inflammatory drugs, pilocarpine, and cevimeline.

Therapeutic compositions can be administered to a subject in need in a wide variety of ways. In various embodiments, the therapeutic composition of the invention is administered orally, intraoperatively, intravenously, intravascularly, intramuscularly, subcutaneously, intracerebrally, intraperitoneally, by soft tissue injection, by surgical placement, by arthroscopic placement, or by percutaneous insertion, e.g., direct injection, cannulation or catheterization. Any administration may be a single administration of a therapeutic composition or multiple administrations. Administrations may be to single site or to more than one site in the subject being treated. Multiple administrations may occur essentially at the same time or separated in time.

Subjects to which administration of the pharmaceutical compositions of the invention is contemplated, after diagnosis using the methods of the invention, include, but are not limited to, humans and other primates, mammals including commercially relevant mammals such as non-human primates, cattle, pigs, horses, sheep, cats, and dogs. In one embodiment, the subject is human.

Pharmaceutical compositions may be administered to subjects in need thereof, identified according to the methods of the invention, in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions are administered to a patient by intradermal or subcutaneous injection. In another embodiment, the compositions are preferably administered by i.v. injection.

The therapeutic composition can be incorporated into any formulation known in the art. For example, the therapeutic composition may be incorporated into formulations suitable for oral, parenteral, intravenous, subcutaneous, percutaneous, topical, buccal, or another route of administration. Suitable compositions include, but are not limited to, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful according to the methods of the invention are not limited to the particular formulations and compositions described herein.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions is contemplated include, but are not limited to, humans and other primates, mammals including commercially relevant mammals such as non-human primates, cattle, pigs, horses, sheep, cats, and dogs.

In the method of treatment, the administration of the compositions may be for either “prophylactic” or “therapeutic” purpose. When provided prophylactically, the compositions are provided in advance of any sign or symptom, although in particular embodiments the invention is provided following the onset of at least one sign or symptom to prevent further signs or symptoms from developing or to prevent present signs or symptoms from becoming more severe. The prophylactic administration of the composition serves to prevent or ameliorate subsequent signs or symptoms. When provided therapeutically, the pharmaceutical composition is provided at or after the onset of at least one sign or symptom. Thus, a pharmaceutical composition may be administered either prior to the development of at least one autoimmune disease or disorder after the onset of the disease or disorder.

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out exemplary embodiments of the present invention, and are not to be construed as limiting in any way the remainder of the disclosure.

### Example 1: Abundance and Nuclear Antigen Reactivity of Fecal Immunoglobulin A in Lupus-Prone (SWRxNZB)-F1 Mice

In the present study, the degree of IgA production in the intestine, and the abundance and nAg reactivity of fecal IgA in lupus-prone SNF1 mice has been studied. The relationship between these features and autoimmune progression in male and female mice, and whether gut microbiota has an influence on fecal IgA abundance and nAg reactivity is studied. These studies, for the first time, show not only that higher amounts of IgA are produced in the gut mucosa associated immune tissues of lupus-prone mice, but also that these antibodies have significant nAg reactivity, even at juvenile age. Correspondingly, the abundance of IgA is profoundly higher in the feces of lupus-prone mice, females particularly, and these antibodies show significant nAg (dsDNA and nucleohistone) reactivity. These fecal IgA features of pre-clinical stages correlate significantly with the eventual systemic autoantibody levels and the proteinuria onset in lupus-prone mice, suggesting that fecal IgA features could be valuable for predicting systemic autoimmune progression prior to seropositivity and lupus nephritis.

The materials and methods used in these experiments are now described

### Mice

SWR/J (SWR), NZB/BlNJ (NZB), C57BL/6J (B6) mice were purchased from the Jackson Laboratory (Bar Harbor, Me.) and housed under SPF conditions. (SWRxNZB)F1 (SNF1) hybrids were generated at the SPF facility of MUSC by crossing SWR females with NZB males. In some experiments, SNF1 mice were given a broad spectrum antibiotic cocktail as described previously (Gudi et al., 2019, Immunology, 157:70-85) to deplete the gut microbiota. Depletion of gut microbiota was confirmed by culture of the fecal pellet suspension on blood agar and brain heart infusion agar plates, under aerobic and anaerobic conditions, as described previously (Gudi et al., 2019, Immunology, 157:70-85). Urine and tail vein blood samples were collected at different time-points to detect proteinuria and autoantibodies.

### Proteinuria

Urine samples were tested weekly for proteinuria. Protein level in the urine was determined by Bradford assay (BioRad) against bovine serum albumin standards. Proteinuria was scored as follows; 0: 0-1 mg/ml, 1: 1-2 mg/ml, 2: 2-5 mg/ml, 3: 5-10 mg/ml and 4: ≥10 mg/ml. Mice that showed proteinuria >5 mg/ml were considered to have severe nephritis.

### ELISA

Antibodies against nAgs (nucleohistone and dsDNA) in mouse sera were evaluated by ELISA. Briefly, 0.5 μg/well of nucleohistone (Sigma-Aldrich) or dsDNA from calf thymus (Sigma-Aldrich) was coated as antigen, overnight, onto ELISA plate wells. Serial dilutions of the sera were made and total IgG against these antigens were detected using HRP-conjugated respective anti-mouse antibody (Sigma-Aldrich, eBioscience and Invitrogen) and the reaction was detected using TMB substrate (BD biosciences). For determining antibody levels in fecal samples, extracts of fresh fecal pellets from individual mice were collected separately and used. Weighed fecal pellets were suspended in proportionate volume of PBS (5% suspension or 50 mg feces/ml; w/v) by breaking the pellet using a pipette tip, high speed vortexing, and continuous shaking at 800 rpm overnight at 4° C. Suspensions were centrifuged at 14,800 rpm for 15 minutes and top ⅔ of the supernatants were collected. Supernatants were diluted 1:100 for determining total IgA concentration. Total IgA levels were determined by employing in-house quantitative sandwich ELISA. Briefly, purified anti-mouse IgA monoclonal antibody (0.1 μg/well in 50 μl) coated wells were incubated with diluted samples for 2 hours, incubated with biotin linked polyclonal anti-mouse IgA antibody for 1 hour, and finally with avidin-HRP for 30 min before developing the reaction using TMB substrate. Purified mouse IgA (Southern biotech) was used in each plate for generating the standard curve. In initial assays to test anti-dsDNA and anti-nucleohistone reactivity of IgA in different sample types, optimum dilution (1:10) of aforementioned extracted samples were incubated in dsDNA or nucleohistone coated plates and further incubated with anti-mouse IgA-HRP before developing the assay. In ELISA assays where dsDNA or nucleohistone antibody titers were determined, serial dilutions of fecal extracts (starting at 1:10 dilution) were incubated in dsDNA or nucleohistone coated plates, incubated with biotin linked anti-mouse IgA antibody, and followed by avidin-HRP. Known positive and negative control samples identified from the initial screening were used in all plates to validate the results for determining a reliable titer. Highest dilution of the sample that produced an OD value of ≥0.05 above background value was considered nAg reactive titer. Per gram IgA concentrations and nAg reactive titers were then calculated for the data presented here.

### Immune Cell Culture

Male and female SNF1 mice, and female B6 mice of 4 and 16 weeks of age were euthanized, single cell suspensions of spleen and Peyer's patches (PP) or enriched immune cells from ileum portion of the small intestine (Si) and large intestine (Li) were prepared as previously described (Johnson et al., 2015, Clin Exp Immunol, 181:323-37; Johnson et al., 2020, Journal of Autoimmunity, 108:102420; Gaudreau et al., 2015, Clinical and experimental immunology, 180:393-407). These cells were subjected to flow cytometry to detect IgA+B cells as described before (Gaudreau et al., 2015, Clinical and experimental immunology, 180:393-407), and/or cultured in the presence of bacterial LPS (2 μg/ml) and anti-CD40 antibody (5 μg/ml) for 48 hours. Optimally diluted spent media were tested for relative levels of total IgA and nucleohistone and dsDNA reactive IgA by ELISA as described above.

### Statistical Analysis

GraphPad Prism or Microsoft excel was used to calculate the statistical significance. Two-tailed t-test or Mann-Whitney test was employed to calculate p-values when two means were compared. Proteinuria scores were analyzed using the Chi-square test. Pearson's correlation coefficient/bivariate correlation approach was employed for measuring linear correlation between two variables. For correlating concentration and titer of two specific factors, Spearman correlation approach was employed. A P value ≤0.05 was considered statistically significant.

The results of the experiments are now described

### Higher Amounts of IgA are Produced in the Gut Mucosa of Lupus-Prone SNF1 Mice

SNF1 mice that develop lupus symptoms and proteinuria spontaneously and show gender bias in disease incidence, similar to human SLE patients (Gaudreau et al., 2015, Clinical and experimental immunology, 180:393-407; Gavalchin et al., 1985, Journal of immunology, 134:885-94; Kalled et al., 1998, Journal of immunology, 160:2158-2165; Annacker et al., 2005, The Journal of experimental medicine, 202:1051-1061), have been widely used for understanding the disease etiology. Significant amounts of circulating autoantibodies against nucleohistone and dsDNA are detectable in these mice by 16 weeks of age and severe nephritis indicated by high proteinuria after 20 weeks of age (Johnson et al., 2015, Clin Exp Immunol, 181:323-37; Gaudreau et al., 2015, Clinical and experimental immunology, 180:393-407). While the timing of detectable levels of serum autoantibodies as well as proteinuria are highly heterogeneous in these mice, about 80% of the female and 20% of the male SNF1 mice develop severe nephritis within 32 weeks. Gut mucosa of female SNF1 mice, as compared to their male counterparts, harbor higher frequencies of activated B cells including plasma cells, as well as express high levels of pro-inflammatory cytokines as early as at juvenile age (4 weeks) and amplified levels of these factors at adult ages (Gaudreau et al., 2015, Clinical and experimental immunology, 180:393-407).

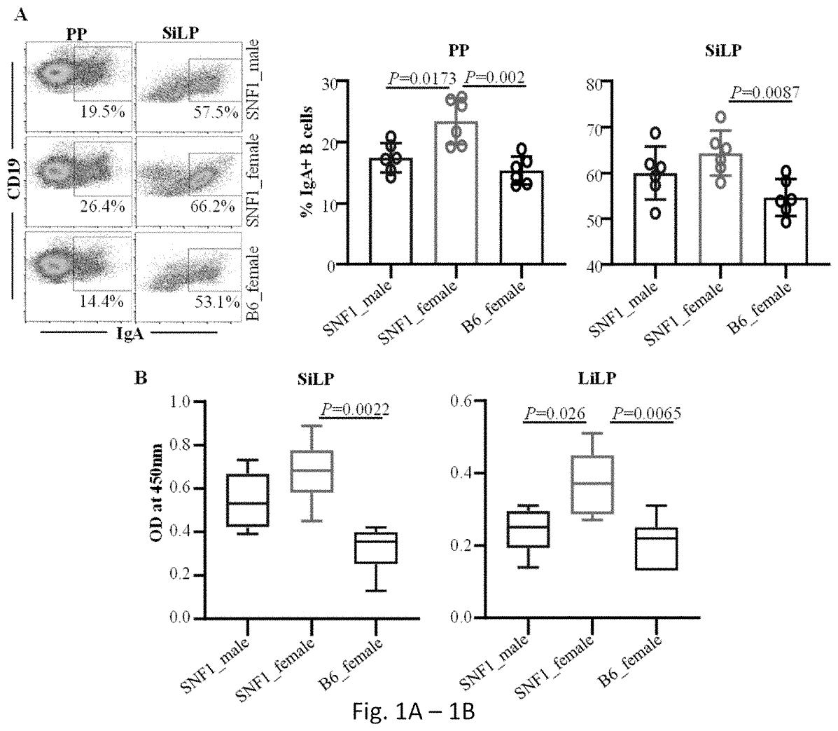

Experiments were conducted to determine whether the degree of IgA production in the gut relates to the pro-inflammatory immune phenotype of gut mucosa. Examination of the frequencies of B cells and IgA+B cells revealed that SNF1 mice have relatively higher occurrence of total and IgA+B cells in the intestinal compartment compared to lupus resistant B6 mice (FIG. 1A). Further, IgA+B cell frequencies, in the intestinal mucosa particularly, were relatively higher in SNF1 females compared to their female counterparts. To assess if the overall amounts of IgA produced in gut mucosa is different in lupus-prone and -resistant mice, total immune cell preparations from the intestinal tissues were tested for spontaneous IgA release. As observed in FIG. 1B, total IgA secreted by the small and large intestinal immune cells was significantly higher in SNF1 mice compared to that of B6 mice. As compared to that of B6 females, cultures of gut associated immune cells of SNF1 mice showed relatively higher age-dependent elevated production of IgA. Comparison of IgA levels in the culture of immune cells from male and female SNF1 mice revealed relatively higher IgA production by gut mucosa associated cells of females than that of their male counterparts. Overall, these observations indicate that the higher degree of IgA production from the gut mucosa of lupus-prone SNF1 mice, females particularly, is reflective of the pro-inflammatory immune phenotype of their gut mucosa, which was described in recent reports (Johnson et al., 2020, Journal of Autoimmunity, 108:102420; Gaudreau et al., 2015, Clinical and experimental immunology, 180:393-407).

### IgA PProduced by the Gut Mucosa Shows nAg Reactivity

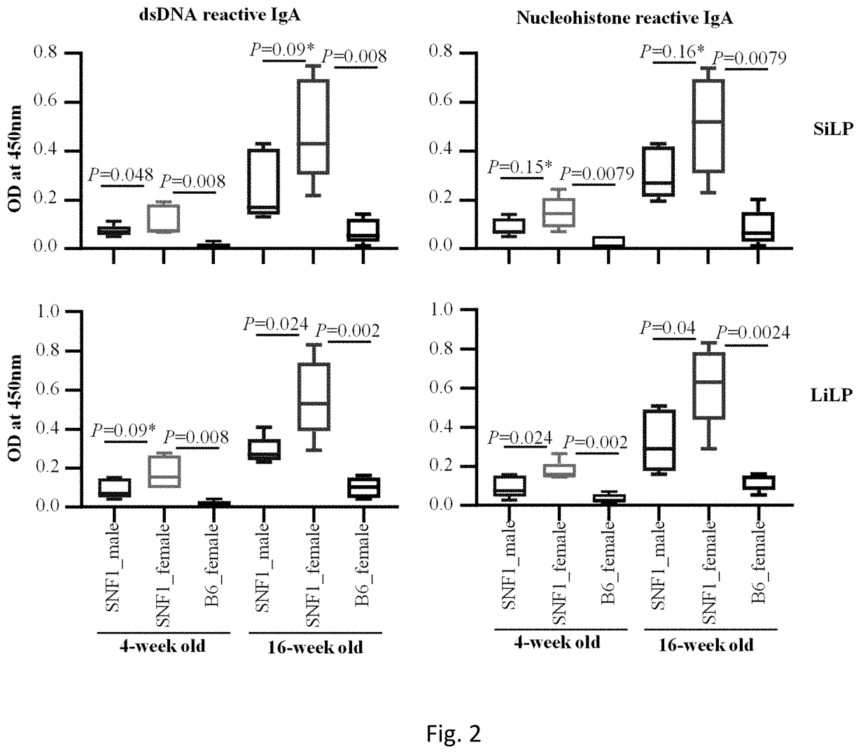

Since cultures of gut mucosa associated immune cells from lupus-prone SNF1 mice showed higher IgA levels compared to that of B6 mice, it was examined if IgA produced in the gut mucosa of juvenile and adult age pre-nephritic mice have nAg reactivity. SiLP and LiLP cells were cultured and the supernatants were examined for nAg (dsDNA and nucleohistone) reactive IgA levels. As observed in FIG. 2, spent media from gut associated immune cell cultures of both juvenile and adult SNF1 mice had higher dsDNA and nucleohistone reactive IgA levels compared to that of B6 mice. Importantly, dsDNA and nucleohistone reactivity of IgA in culture of gut associated immune cells from female SNF1 mice were significantly higher compared to that of their male counterparts at both juvenile and adult ages. Overall, these observations show that IgA produced in the gut mucosa of lupus-prone mice, as early as juvenile age, can recognize nAg and the degree of nAg reactivity correlates with the known gender bias of lupus incidence in this mouse model.

### SNF1 Mice Show Higher Abundance of IgA in the Feces

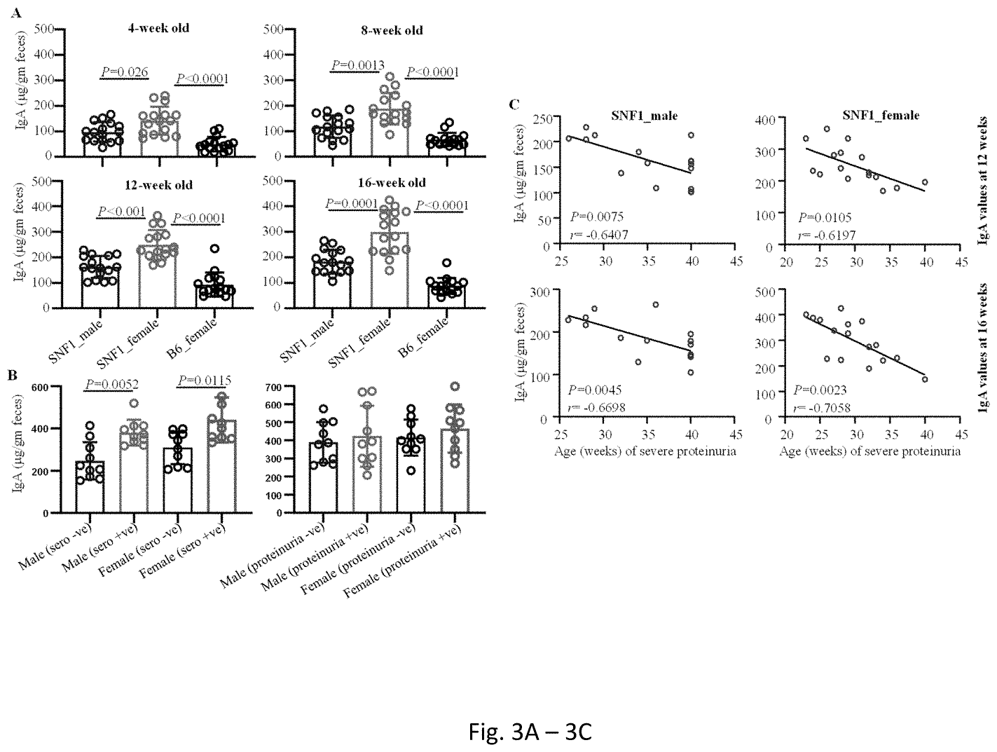

Since gut associated immune cell cultures showed higher levels of IgA antibody, fecal samples from lupus-prone male and female SNF1 mice and age matched female B6 mice were examined for IgA levels. Quantification of IgA in fecal samples collected from different age groups of mice revealed that fecal levels of IgA are significantly higher in male and female lupus-prone SNF1 mice at all ages, including juvenile, compared to that of female B6 mice (FIG. 3A). Further, differences in fecal IgA levels in lupus-prone SNF1 mice and lupus-resistant B6 mice were more pronounced in older adults compared to juveniles. Importantly, compared to male counterparts, female SNF1 mice showed relatively higher amounts of fecal IgA as early as at juvenile age.

The fecal IgA levels in age-matched seronegative and seropositive as well as in sero and proteinuria-positive SNF1 males and females are different. FIG. 3B shows that both male and female seropositive SNF1 mice showed significantly higher levels of fecal IgA compared to their seronegative counterparts. Similarly, although not significant comparable to that of seropositive mice, higher fecal IgA levels were observed in sero and proteinuria-positive SNF1 male and females compared to seronegative counterparts. Overall, these observations suggest a positive correlation between fecal IgA levels of pre-seropositive younger ages and the eventual disease onset in SNF1 mice. Therefore, fecal IgA levels of 12 week and 16 week old pre-nephritic mice were correlated with the timing of severe proteinuria. As observed in FIG. 3C, significant correlation between younger age fecal IgA levels and eventual clinical stage of disease is seen, suggesting that fecal IgA levels could serve as a marker for systemic autoimmune progression of pre-clinical stages.

### Anti-dsDNA and Nucleohistone Reactive Fecal IgA Antibodies are Detected in SNF1 Mice

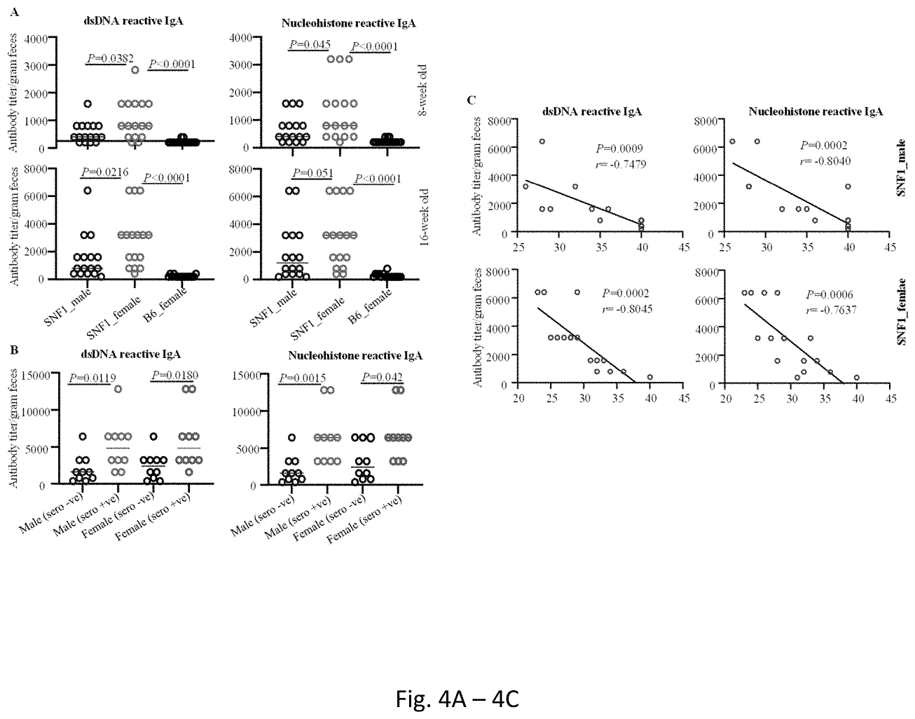

Since fecal IgA levels were higher in lupus-prone SNF1 mice compared to B6 mice and in SNF1 females compared to male counterparts, it was determined whether fecal IgA antibodies from SNF1 mice recognize nuclear antigens such as dsDNA and nucleohistones, and whether this feature correlates with systemic autoimmunity and disease incidence. Compared to age-matched female B6 mice, both male and female SNF1 mice showed significantly higher levels of dsDNA and nucleohistone reactive IgA antibodies in their fecal samples at all ages (FIG. 4A). dsDNA and nucleohistone reactivity of fecal IgA were detectable only in female, but not male, SNF1 mice at juvenile ages. Further, although fecal IgA antibodies of majority of male and female SNF1 mice showed dsDNA and nucleohistone reactive at older ages, higher number of female SNF1 mice had significantly higher levels of nAg-reactive fecal IgA compared to male counterparts at all tested adult ages. As shown in FIG. 3B, nAg reactivity titers of fecal IgA were higher in seropositive male and female SNF1 mice compared to their seronegative counterparts. Overall, these analyses show that nAg reactive IgA antibody can be found in fecal samples long before lupus associated autoantibodies are detected in the systemic compartment. Further, a strong correlation between early age fecal IgA nAg reactivity of both male and female SNF1 mice and the timing of eventual onset of proteinuria was detected (FIG. 3C). These observations suggest that nAg reactivity of fecal IgA at pre-seropositive stages could be predictive of the eventual disease onset in SNF1 mice.

### Depletion of Gut Microbiota Results in Suppression of Fecal IgA Abundance and nAg Reactivity

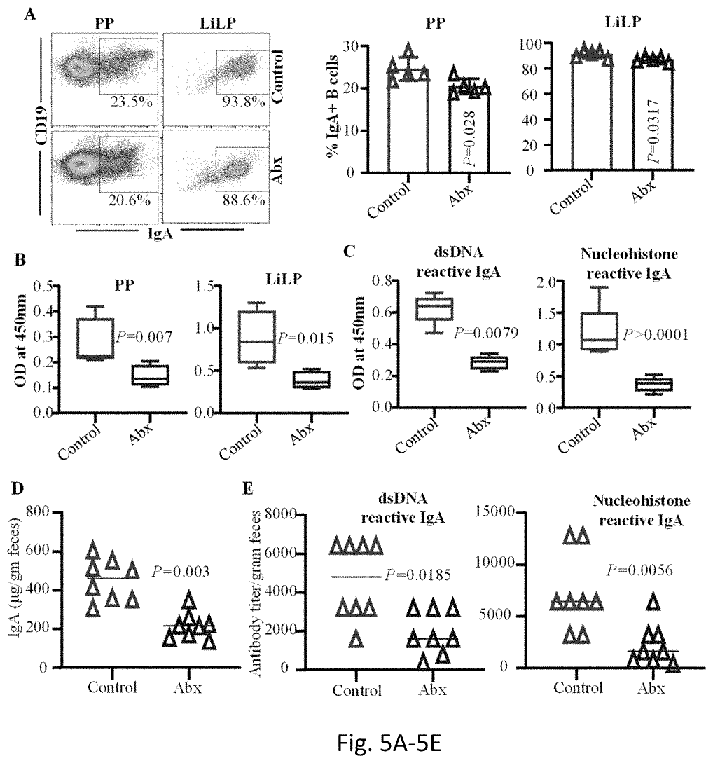

Microbes are the primary trigger of IgA production in the gut mucosa (Hapfelmeier et al., 2010, Science, 328:1705-1709; Peterson et al., 2007, Cell Host Microbe, 2:328-339). Depletion of gut microbiota suppresses intestinal pro-inflammatory immune phenotype in juvenile and adult SNF1 mice, systemic autoimmune progression and proteinuria onset. Therefore, IgA production in the gut mucosa, and abundance and nAg reactivity of fecal IgA are influenced by gut microbiota, was examined by treating the mice with broad spectrum antibiotic cocktail. As observed in FIG. 5A, IgA+B cell frequencies were significantly lower in microbiota depleted mice compared to untreated mice. Further, intestinal immune cell cultures from microbiota depleted mice showed significantly lower amounts of spontaneously released IgA antibodies compared to that of untreated control mice (FIG. 5B). Cultures of intestinal immune cells from microbiota depleted mice showed significantly lower nAg reactive IgA levels compared to that of control mice (FIG. 5C). Correspondingly, FIG. 5D shows that microbiota depletion caused significant reduction in IgA abundance in fecal samples. Moreover, dsDNA and nucleohistone reactive fecal IgA levels were significantly lower in SNF1 mice with depleted microbiota compared to their counterparts with intact gut microbiota. Of note, serum samples from microbiota depleted SNF1 mice have significantly lower anti-dsDNA and nucleohistone IgG antibodies compared to mice with intact microbiota and the disease onset is delayed only in females, but not in males, upon microbiota depletion (Johnson et al., 2020, Journal of Autoimmunity, 108:102420). Overall, these observations indicate that fecal IgA abundance and nAg reactivity in lupus-prone SNF1 mice are, at least in part, microbiota dependent and validate that these features of fecal IgA are reflective of the degree of systemic autoimmune activity.

Immunoglobulin A not only neutralizes pathogens at mucosal surfaces, but also regulates the composition and function of gut microbiota by stabilizing the intestinal colonization by symbiotic microorganisms (Nakajima et al., 2018, The Journal of experimental medicine, 215:2019-2034; Fadlallah et al., 2018, Sci Transl Med, 10; Donaldson et al., 2018, Science, 360:795-800). Recent reports have shown that the degree of IgA production in the gut mucosa under normal and clinical conditions including SLE can be different (Azzouz et al., 2019, Ann Rheum Dis, 78:947-956; Dzidic et al., 2017, J Allergy Clin Immunol, 139:1017-25 e14; Frehn et al., 2014, PloS one, 9:e106750). A recent report has shown that fecal IgA and IgG levels are higher in SLE patients compared to healthy controls (Frehn et al., 2014, PloS one, 9:e106750). Nevertheless, if IgA produced in the gut mucosa contributes to autoimmune process or higher antibody production in the gut mucosa in SLE patients precedes systemic autoimmunity and clinical onset of the disease or is the consequence of autoimmune process is unknown. Further, an association between IgA production in the gut mucosa and gender bias associated with lupus has not been studied. Recent reports (Johnson et al., 2020, Journal of Autoimmunity, 108:102420; Gaudreau et al., 2015, Clinical and experimental immunology, 180:393-407) showed, for the first time, that the immune phenotype of gut mucosa is significantly different in lupus-prone male and female SNF1 mice. Pro-inflammatory immune phenotype of gut mucosa including higher frequencies of plasma cells appear in female SNF1 mice at juvenile age, much before the production of sex hormones and detectable levels of circulating autoantibodies. These observations suggest that production of IgA in the gut mucosa and its levels in feces, may be significantly different in males and females under lupus susceptibility prior to disease onset. In the current study, higher amounts of IgA antibodies were detected in the fecal samples of lupus-prone SNF1 mice and the levels of these antibodies correlate positively with the rapid disease progression and higher disease incidence of female SNF1 mice. Most importantly, nuclear antigen-recognizing fecal IgA antibodies can be detected in lupus-prone mice as early as juvenile age and these fecal antibody levels of younger ages correlate with eventual circulating autoantibody levels and disease progression at adult ages, and the gender.

SNF1 mice that develop lupus symptoms and proteinuria spontaneously and show strong gender bias in disease incidence, similar to human SLE patients (Gaudreau et al., 2015, Clinical and experimental immunology, 180:393-407; Gavalchin et al.,1985, Journal of immunology, 134:885-94; Kalled et al., 1998, Journal of immunology, 160:2158-2165; Annacker et al., 2005, The Journal of experimental medicine, 202:1051-1061), have widely been used for understanding the disease etiology. Significant amounts of circulating autoantibodies against nucleohistone and dsDNA are detectable in these mice by 16 weeks of age and severe nephritis occurs, indicated by high proteinuria, after 20 weeks of age (Johnson et al., 2015, Clin Exp Immunol, 181:323-37; Gaudreau et al., 2015, Clinical and experimental immunology, 180:393-407). While the timing of detectable levels of serum autoantibodies as well as proteinuria are heterogeneous, about 80% of the female and 20% of the male SNF1 mice develop severe nephritis within 32 weeks. Hence, the present observation that IgA levels, although highly heterogeneous, are higher in significant number of lupus-prone females compared to males even at juvenile age, much before the clinical onset of disease, is noteworthy. Notably, fecal levels of IgA at younger ages showed excellent correlation with older adult age proteinuria onset indicating that fecal IgA levels may serve as biomarker for early detection of clinical onset of SLE. These observations, along with recent reports showing pro-inflammatory immune phenotype of gut mucosa including higher amounts of large number of pro-inflammatory and large number of plasma cells appears in female SNF1 mice at juvenile age (Gaudreau et al., 2015, Clinical and experimental immunology, 180:393-407), indicates that pro-inflammatory cytokines and IgA produced in the gut mucosa may be involved in the initiation and perpetuation of systemic autoimmunity in lupus.

It is well established that gut microbiota including symbionts in general, and specific microbial communities including pathobionts in particular, can influence the magnitude of antibody production in the gut mucosa and the IgA abundance in feces (Hapfelmeier et al., 2010, Science, 328:1705-1709; Peterson et al., 2007, Cell Host Microbe, 2:328-339; Planer et al., 2016, Nature, 534:263-266; Lecuyer et al., 2014, Immunity, 40:608-620; Macpherson et al., 2001, Microbes Infect, 3:1021-1035). Interestingly, however, gut microbiota composition is significantly different in lupus-prone males and females only at adult age, but not at juvenile age (Johnson et al., 2020, Journal of Autoimmunity, 108:102420), suggesting that the production of higher pro-inflammatory cytokines and IgA in the gut mucosa at younger ages is not due to differences in the gut microbiota. It is possible that differences in the types and degrees of host-microbe molecular interactions at gut mucosa may be responsible for differences in the immune phenotypes of lupus-prone males and females. Irrespective of the mechanisms involved, pro-inflammatory features including hyper-activation of B cells of the gut mucosa in the presence of gut microbial components could aid in the production of anti-microbial antibodies that cross react with host antigens. In this regard, microbial antigens are continuously sampled by immune cells of the gut mucosa and it can result in local and systemic responses (Rios et al., 2016, Mucosal Immunol, 9:907-916; Schulz et al., 2013, Trends in immunology, 34:155-61; Stagg et al., 2003, Gut, 52:1522-1529; Zhao et al., 2018, Immunology, 154:28-37). Further, host-microbial interactions could aid in autoimmune initiation and progression in at-risk subjects, via multiple mechanisms including bystander activation and molecular mimicry (Belkaid et al., 2014, Cell, 157:121-141; Theofilopoulos et al., 2017, Nature immunology, 18:716-724; Haase et al., 2018, Immunology, 154:230-238). In fact, contribution of molecular mimicry, primarily by antigens of pathogenic bacteria, in the initiation and/or perpetuation of T and B cell responses to self-antigens have been widely investigated (Shimoda et al., 1995, The Journal of experimental medicine, 181:1835-1845; Chastain et al., 2012, Immunological reviews, 245:227-238). A recent report has shown that pathogenic autoreactive T and B cells can cross react with mimotopes expressed by a human gut commensal and contribute to autoimmunity in an anti-phospholipid syndrome (APS) model (Ruff et al., 2019, Cell Host Microbe, 26:100-13 e8).

Autoantibody response against nuclear antigens such as nucleic acids and nuclear proteins including histones, is the key feature of lupus autoimmunity in humans and rodent models. Importantly, nucleic acids and histone-like and other nucleic acid binding proteins can be structurally homologous to their mammalian host counterparts (Gilkeson et al., 1989, Journal of immunology, 142:1482-1486; Pisetsky et al.,1990, Arthritis and rheumatism, 33:153-159; Sabbatini et al., 1993, European journal of immunology, 23:1146-1152; Balandina et al., 2002, The Journal of biological chemistry, 277:27622-27628; Kamashev et al., 2017, PloS One, 2017;12:e0188037), suggesting that cross-reactive antibodies against microbial and host molecules are generated in the gut mucosa. This also prompts the notion that antibody production in the gut mucosa against these microbial components could eventually spread to the systemic compartment in lupus-prone background as autoimmunity against host nuclear antigens. It is possible that pro-inflammatory gut mucosa of lupus-susceptible background, as observed in lupus-prone mice (Johnson et al., 2020, Journal of Autoimmunity, 108:102420; Gaudreau et al., 2015, Clinical and experimental immunology, 180:393-407; Esposito et al., 2014, Clin Microbiol Infect Dis, 33:1467-1475), could facilitate this process. In fact, the detection of fecal IgA in lupus-prone female SNF mice that are dsDNA and nucleohistone reactive as early as juvenile age, much earlier than the appearance of circulating autoantibodies, supports this notion. Importantly, age-dependent progressive increase in the dsDNA and nucleohistone reactive fecal IgA and strong correlation between the levels of these antibodies at younger ages and the eventual systemic autoimmune progression was observed in this study. This suggests a vicious cycle of pro-inflammatory responses in the gut mucosa contributing to the generation of a large number of host nuclear antigen and microbial antigen cross-reactive B cells in the gut mucosa and their eventual spread to the systemic compartment leading to the pathogenic events of systemic autoimmunity in lupus.

IgA-antibody production in the gut mucosa is largely influenced by microbiota and the GF mice as well as microbiota depleted SPF mice show significantly lower amounts of fecal IgA (Planer et al., 2016, Nature, 534:263-266; Bunker et al., 2018, Immunity, 49:211-224; Wilmore et al., 2018, Cell Host Microbe, 23:302-11 e3; Robak et al., 2018, The Journal of clinical investigation, 128:3535-3545). Gut microbial communities including symbionts and pathobionts could differently impact the degree of IgA production in the gut mucosa (Peterson et al., 2007, Cell Host Microbe, 2:328-339; Gutzeit et al., 2014, Immunological reviews, 260:76-85; Isobe et al., 2020, Int Immunol; 32:243-258; Karnada et al., 2013, Nature immunology, 14:685-690). The data presented herein demonstrate that depletion of gut microbiota using broad spectrum antibiotics not only caused the suppression of fecal total IgA levels, but also the degree of dsDNA and nucleohistone reactivity.

Overall, this study demonstrates, for the first time, that fecal IgA profiles are different in lupus-prone SNF1 mice and lupus-resistant B6 mice as early as juvenile age and this feature is reflective of late adult age systemic autoimmunity and nephritis. Most importantly, fecal IgA in lupus-prone mice show nAg reactivity at younger ages, long before the detection of systemic autoantibodies and lupus nephritis. Furthermore, fecal IgA abundance and nAg reactivity correlate with the lupus-like disease associated gender bias in this mouse model. These novel observations suggest that fecal IgA levels and nuclear antigen reactivity could serve as biomarker to detect the systemic autoimmune activities in at-risk subjects, long before the disease onset. Since the abundance and nAg reactivity of fecal IgA in lupus-prone subjects have not been studied, systematic longitudinal follow-up studies are needed to determine the clinical translation value of IgA features as biomarkers to predict the clinical disease.

### Example 2: Autoreactive Fecal Immunoglobulin A (IgA) as Biomarker for Lupus

Recent preclinical studies using a spontaneous model of SLE (Johnson et al., 2015, Clin Exp Immunol, 181:323-37; Johnson et al., 2020, Journal of Autoimmunity, 108:102420; Gaudreau et al., 2015, Clinical and experimental immunology, 180:393-407) and preliminary results show that proinflammatory events including autoantibody production initiated in the gut mucosa at juvenile age, long before the detection of systemic autoimmunity, are the initiators and perpetuators of systemic autoimmune process in lupus. In juvenile age lupus-prone NZBxSWR-F1 (SNF1) female mice: 1) microbiota dependent pro-inflammatory cytokine expression has been observed in the gut mucosa, which is suppressed upon treatment with broad spectrum antibiotics (Johnson et al., 2020, Journal of Autoimmunity, 108:102420); 2) pro-inflammatory cytokine secreting B cells have been observed in gut associated lymphoid tissues (Gaudreau et al., 2015, Clinical and experimental immunology, 180:393-407), 3) gut origin of autoantibody production has been observed, as indicated by juvenile age production of autoantibodies in the intestinal mucosa, but not in the systemic compartment.

Importantly, the data presented herein demonstrates that the amounts of IgA, the most abundant immunoglobulin (Ig) type released into the gut lumen, are higher in the fecal samples of lupus-prone mice long before the detection of clinical stage disease. Furthermore, fecal IgA of these lupus-prone mice at pre-clinical stages show profound anti-dsDNA and nucleohistone reactivity. In addition, significantly higher amounts and anti-dsDNA reactivity of IgA is observed in stool samples of SLE patients. Based on these novel observations, the data demonstrates that self-antigen reactive antibody production in SLE is initiated in the gut mucosa, and the abundance and nuclear antigen (nAg) reactivity of fecal IgA at preclinical stages can serve as biomarkers to predict the autoimmune progression and disease onset.

### Determine the Dynamics of Intestinal IgA Production, Fecal IgA-Abundance and -nAg Reactivity, and the Correlation of these Features with Systemic Autoimmune Progression

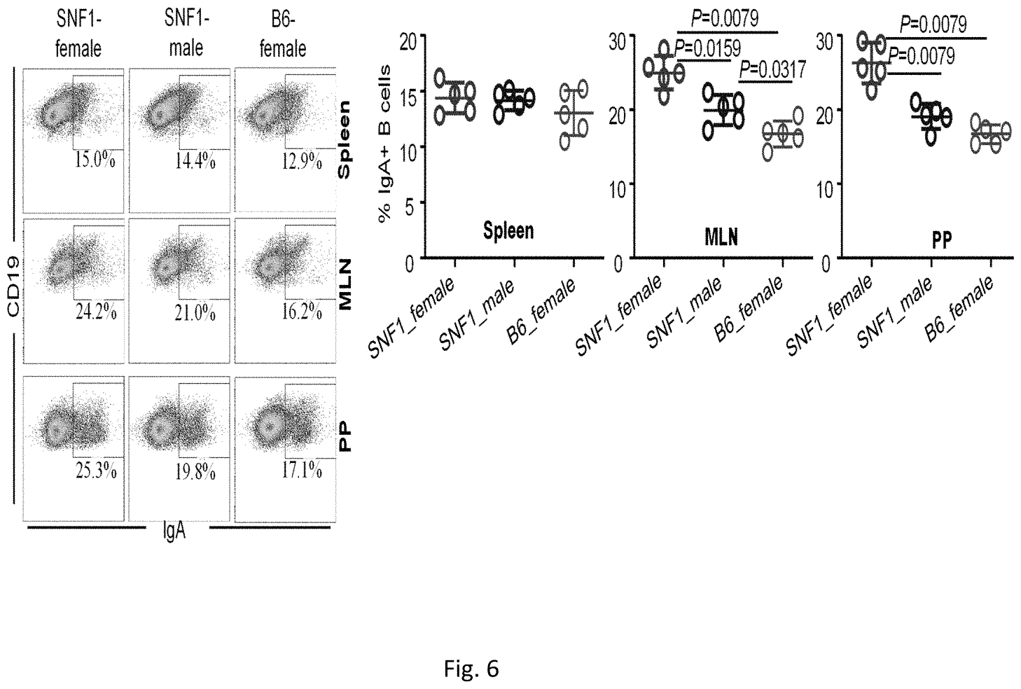

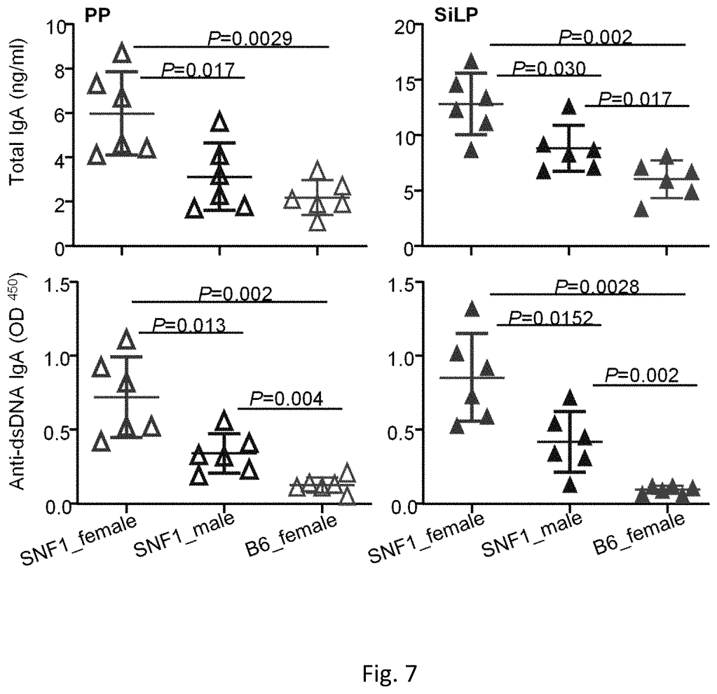

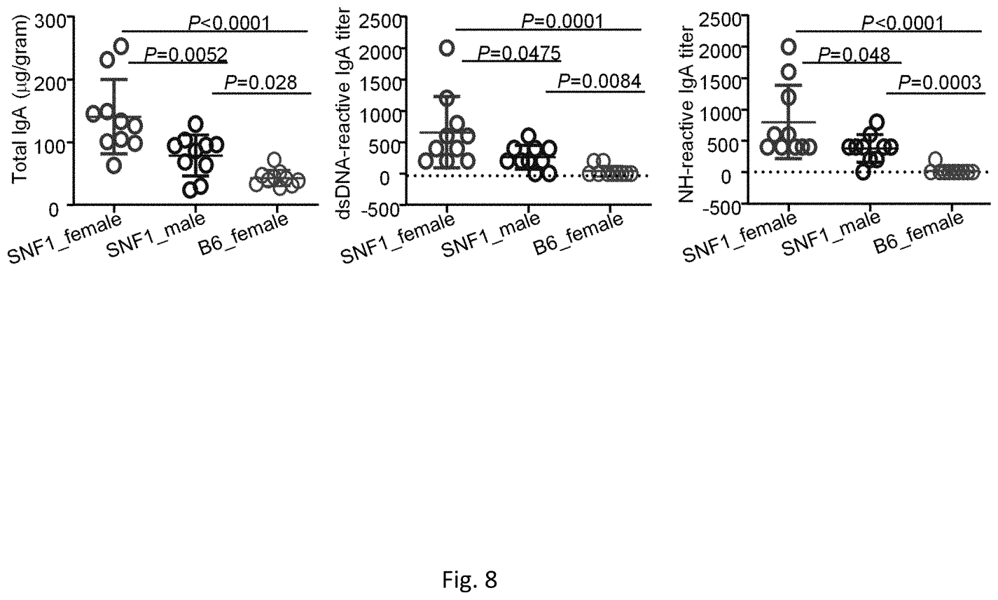

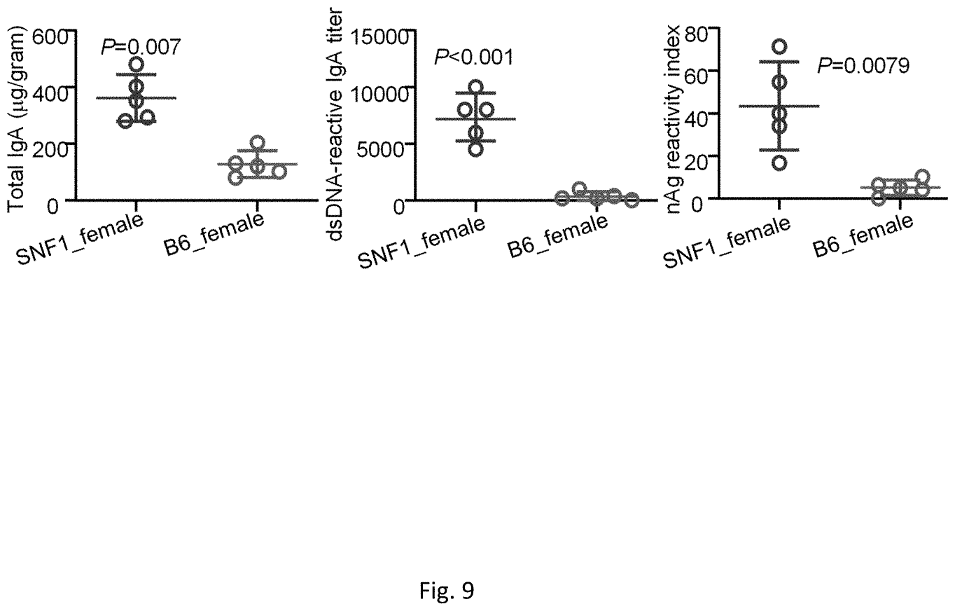

Rapid disease progression and higher disease incidence in lupus-prone female SNF1 mice (F1 generation of SWRxNZB crossing) correlate with the microbiota dependent proinflammatory phenotype of gut mucosa at juvenile age (Johnson et al., 2020, Journal of Autoimmunity, 108:102420; Gaudreau et al., 2015, Clinical and experimental immunology, 180:393-407). In SNF1 mice, high-titer circulating autoantibodies and proteinuria can be detected in females starting about 16-wks and 22-wks of age respectively, and >90% females and about 30% males develop severe nephritis, as indicated by high proteinuria, by 35-wks of age (Johnson et al., 2015, Clin Exp Immunol, 181:323-37; Johnson et al., 2020, Journal of Autoimmunity, 108:102420; Gaudreau et al., 2015, Clinical and experimental immunology, 180:393-407). This suggests that pro-inflammatory features of gut mucosa in these mice appear long before the appearance of systemic antibodies and clinical stage disease. Since it is likely that proinflammatory cytokines could activate gut resident B cells and facilitate the production of IgA antibody, the frequencies of IgA+ plasma cells were assessed in the gut mucosa of SNF1 mice at sero-negative age. As observed in FIG. 6, IgA+ plasma cell frequencies are significantly higher in the Peyer's patches (PP) and mesenteric lymph nodes (MLN), but not in the spleen, of 8-Wk old SNF1 females compared to age-matched males and lupus-resistant female B6 mice. The degree of IgA production by intestinal B cells was determined in another set of mice. Upon short-term ex vivo stimulation, PP and small intestinal lamina propria (SiLP) cells from 8-week old female SNF1 mice produced higher amounts of IgA antibodies compared to these cells from SNF1 males and B6 females (FIG. 7). Since autoantibodies in human SLE and the mouse models bind to nAgs including dsDNA and histone, whether IgA produced by these cells of seronegative age SNF1 mice have nAg reactivity was examined. As observed in FIG. 7, while cells from both male and female SNF1 mice produced higher anti-dsDNA IgA compared to B6 mouse cells, cells from female SNF1 mice produced higher amounts of anti-dsNA antibodies compared to these cells from male SNF1 mice. These observations demonstrate that fecal IgA-levels and -nAg reactivity could be significantly higher in pre-seropositive female SNF1 mice compared to their male and lupus-resistant female B6 counterparts. As observed in FIG. 8, IgA concentration in fecal pellets was significantly higher in 12-week old SNF1 females, compared to their male counterparts. Furthermore, both dsDNA and nucleohistone reactive IgA levels were higher in most SNF1 females compared to males and profoundly higher when compared to B6 mice. Moreover, the abundance and nAg reactivity of fecal IgA were profoundly higher in older sero- and proteinuria-positive SNF1 mice compared to age matched B6 mice (FIG. 9). Overall, these observations suggest that higher amounts of IgA are produced in the gut mucosa of young lupus-prone mice and these antibodies possess nAg reactivity long before the detection of systemic autoimmunity and clinical disease. The data suggests that fecal IgA features (abundance and/or nAg reactivity) can serve as a biomarker for early detection of systemic autoimmunity in lupus at-risk subjects. The dynamics of IgA production in the gut mucosa, and the abundance and nAg reactivity of fecal IgA at pre-clinical stages are determined in a longitudinal study using this spontaneous, slow-progressing, autoimmune lupus model of SNF1 mice. Features of intestinal and fecal IgA at as early as juvenile age are associated with eventual systemic autoimmune progression including the appearance of circulating autoantibodies and nephritis/proteinuria.

The SNF1 mouse model has been used in previous studies (Johnson et al., 2015, Clin Exp Immunol, 181:323-37; Johnson et al., 2020, Journal of Autoimmunity, 108:102420; Gaudreau et al., 2015, Clinical and experimental immunology, 180:393-407) primarily because this strain, which develops spontaneous lupus, shares a large number of histone epitopes with SLE patients and show slow disease progression as well as gender bias as in humans (Xie et al., 2002, Genes Immun, 3 Suppl 1:S13-20; Ghatak et al., 1987, Proc Natl Acad Sci U S A 84, 6850-3; Kang et al., 2011, J Clin Immunol 31:379-394; Gavalchin et al.,1928, Journal of immunology, 134:885-94; Kalled et al., 1998, Journal of immunology, 160:2158-2165; Eastcott et al., 1983, J Immunol 131:2232-2239; Kaliyaperuma et al., 1996, J Exp Med 183, 2459-69; Kaliyaperuma et al., 2002, J Immunol 168:2530-2537; Lu et al., 1999, J Clin Invest 104:345-355). Hence, different age and disease stage groups of this strain of lupus-prone mice, both male and female, along with age matched lupus-resistant female B6 control mice are used in this study. First, the dynamics of IgA production is assessed in the gut mucosa by examining GALT cells from mice at different ages for the frequencies of IgA+ plasma cell and the degree of IgA secretion by them. Second, progressive changes in the IgA concentration in fecal pellets, collected from individual mice at timely intervals, are determined by employing mouse IgA quantitative ELISA. NAg reactivity of IgA is examined in these samples by using anti-dsDNA and -nucleohistone IgA ELISA assays. These mice are monitored for the appearance of circulating autoantibodies (sero-positivity) and proteinuria (nephritis) to correlate the appearance of these disease associated features with fecal IgA-features.

### Dynamics of IgA+ Plasma Cells and IgA Production in the Gut Mucosa

The following age groups of male and female SNF1 mice are euthanized along with age-matched male and female B6 mice, GALT [PP, distal small intestine/ileum (Si), and large intestine/colon (Li)], mesenteric LN (MLN) and systemic control tissues (spleen and blood) are collected for determining IgA+ plasma cell frequencies and antibody production: 1) 4-wk old (juvenile) (no detectable level of circulating anti-nAg Abs) 2) 8-wk old (adult) (no detectable level of circulating anti-nAg Abs) 3) 12-wk old (Increase in circulating anti-nAg IgG in <10% females) 4) 16-wk old (high levels of circulating anti-nAg IgG in about 40% of females and <10% of males) 5) 24-wk old (high levels of anti-nAg IgG in >80% females and about <30% males; about 20% females with severe nephritis/proteinuria). Single cell suspension are prepared from PP, MLN and spleen. PBMCs are prepared from blood collected in anticoagulant upon euthanasia. Immune cell enriched fractions are also prepared from SiLP and LiLP by collagenase digestion followed by Percoll gradient centrifugation as described in recent reports (Johnson et al., 2020, Journal of Autoimmunity, 108:102420; Gaudreau et al., 2015, Clinical and experimental immunology, 180:393-407; Gudi et al., 2019, Immunology 157, 70-85; Sofi et al., 2019, Diabetes 68, 1975-1989). Although large number of IgA+ plasma cells are expected, primarily in the GALT, spleen and blood is also tested to determine 1) the abundance of these cells in circulation and the systemic lymphoid organ, 2) the production of nAg reactive Abs, and 3) an age-dependent progressive increase in frequencies. Cells are stained for CD19 (B cell marker), CD138 (plasma cell marker) and IgA, and subjected to FACS analysis to determine IgA+ plasma cell frequencies. Equal number of cells from these preparations from different groups are cultured for 24 and 48 hour time-points in the presence of bacterial LPS and anti-CD40 Ab, supernatants are tested for the amounts of total IgA by quantitative sandwich ELISA to realize the degree of IgA secretion. For some assays, B cells are enriched (from SiLP, LiLP, MLN and spleen cells particularly) using magnetic sorting targeting CD19, and equal number of cells are activated. Secreted IgA levels are determined in these samples.

Supernatants are also tested for the nAg reactive IgA levels by indirect ELISA using calf thymus dsDNA and nucleohistone coated plates. IgA+ plasma cell frequencies, total IgA -abundance and -nAg reactivity of female SNF1 mouse cells are compared with that of SNF1 males and B6 females. Further, age-dependent changes, correlation with the disease features (circulating anti-nAg IgG and proteinuria onset and severity) are also assessed for older age groups. These studies demonstrate that there is a progressive increase in the production of nAg reactive and total-IgA in the gut, and these features of younger mice correlate with systemic autoimmunity of older mice. These studies also show the degree of nAg reactive IgA production by B cells of intestine is higher than that by spleen cells at each time-point.

### Fecal IgA Level and nAg Reactivity

To determine fecal IgA levels, fresh fecal pellets from a large cohorts of male and female SNF1 mice and control male and female B6 mice are collected (individually from each mouse) at timely intervals, starting at weaning age (3-Wk, 4-Wk, 8-Wk, 12-Wk, 16-Wk, 20-Wk, 24-Wk, 28-Wk and 32-Wk). Fecal pellets are weighed, homogenized in PBS and the soluble fractions are separated by high speed centrifugation. Total IgA concentration is determined in optimally diluted extracts by quantitative sandwich ELISA. The IgA concentration/gram of fecal pellet is calculated and compared between groups, and within individual mice and groups at different ages. The nAg reactivity titer of fecal IgA is tested in serial dilutions of these samples by dsDNA and nucleohistone ELISA assays. ELISA nAg reactive titer values of fecal IgA (highest dilution with ≥0.1 OD above the background) are converted to titer/gram feces, and then compared to that between groups, and within individual mice and groups at different time-points as for total IgA values. To validate the nAg reactivity of fecal IgA determined by ELISA further, standard antinuclear autoantibody (ANA) immunofluorescence assay (IFA) using Hep2 cell substrate slides (Bio-Rad) and Alexa Fluor 488 linked anti-mouse IgA are carried out for serial dilutions of fecal extracts, and the ANA titer is determined. IFA ANA-titer values are compared within and between the groups as for ELISA values. To calculate the nAg reactivity index (NRI) relative to total IgA concentration, dsDNA and nucleohistone reactive titers of each samples are divided by total IgA concentration. This determines the true extent of secretion of nAg reactive-IgA in the feces of lupus-prone mice, compared to lupus-resistant B6 mice. To determine the nAg reactivity of fecal IgA of lupus-prone mice specific to DNA and nuclear proteins or non-specific poly-reactivity, all samples are also tested for non-specific ovalbumin and dinitrophenyl (DNP) reactivity. To determine whether the higher fecal IgA level in lupus-prone mice is directed against gut microbes rather than self-antigen, lysates of fecal microbial preparation, enriched and washed in acidic buffer to remove bound native IgA, as well as E. coli lysate are employed as antigens in ELISA. Use of these control antigens helps determine if nAg reactivity of fecal IgA of lupus-prone mice is specific. Overall, these analyses demonstrate that age dependent increase in total and nAg reactive IgA occur in lupus-susceptible and resistant mice, and that systemic autoimmune progression in lupus-prone mice correlates with these features of fecal IgA. To correlate fecal IgA -levels and -nAg reactivity with systemic autoimmune progression and nephritis, aforementioned male and female SNF1 mice and male and female B6 mice are monitored systematically for up to 40 weeks of age for proteinuria and circulating anti-nAg (dsDNA and nucleohistone) IgG levels (Johnson et al., 2015, Clin Exp Immunol, 181:323-37; Johnson et al., 2020, Journal of Autoimmunity, 108:102420; Gaudreau et al., 2015, Clinical and experimental immunology, 180:393-407). Urine samples are collected weekly starting at week 3 of age and tested for protein levels employing Bradford method to detect nephritis. Serum samples (from tail vein blood) collected every two weeks starting 4 weeks are examined for anti-dsDNA and -nucleohistone IgG titer by ELISA to determine systemic autoimmune onset and progression. Mice are euthanized upon showing high proteinuria (>5 mg/ml) for two consecutive weeks or upon termination of the experiment at 40 weeks of age, H&E stained kidney sections are examined to confirm the proteinuria results on nephritis. The seronegative age fecal IgA-levels and -nAg reactivity of each mouse and each group are correlated separately with its timing of sero-positivity and proteinuria onset. The degree of these correlations of female SNF1 vs their male counterparts is compared to determine the gender bias in the fecal IgA features in lupus.

### Levels and nAg Reactivity of Fecal IgA in Human SLE

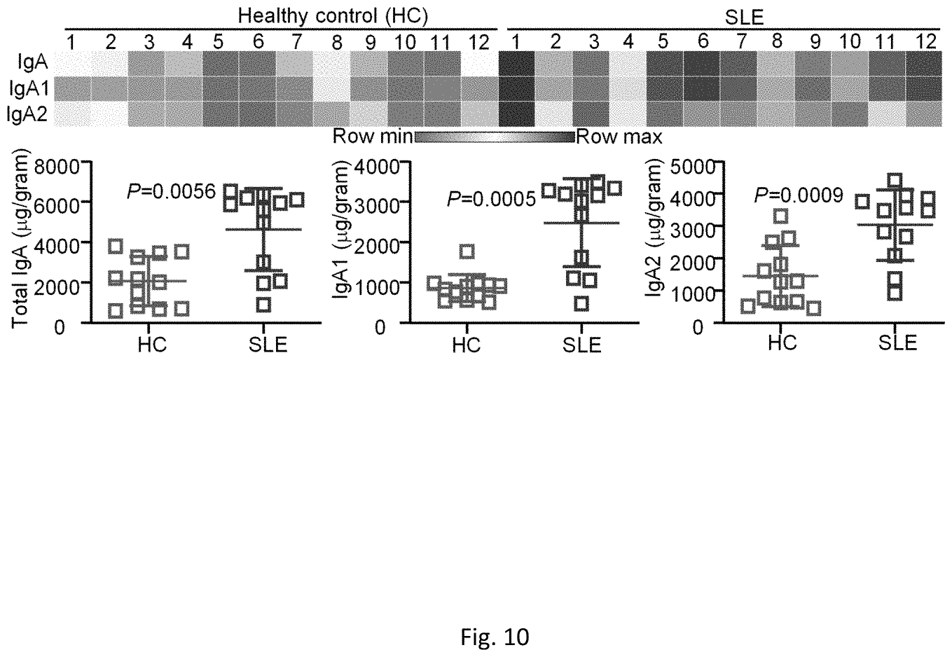

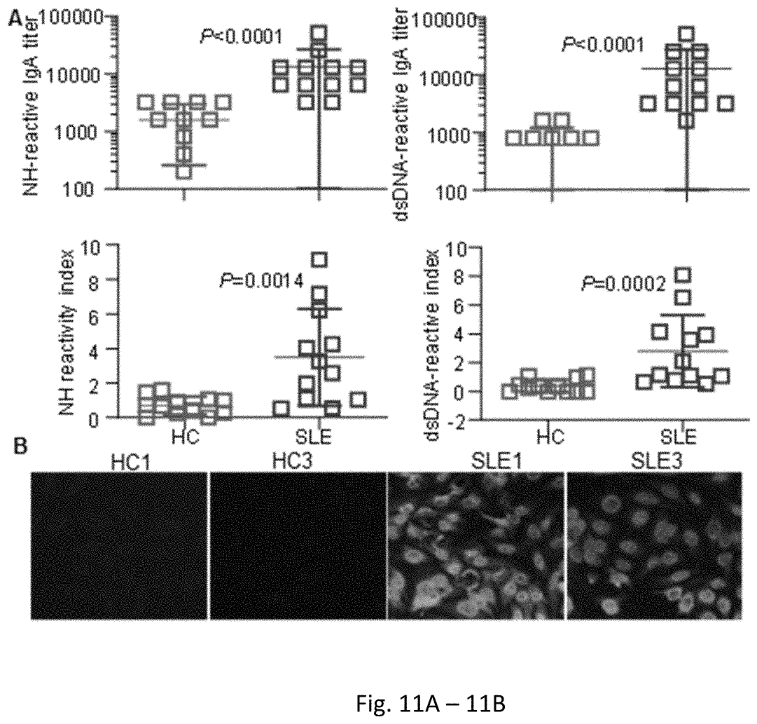

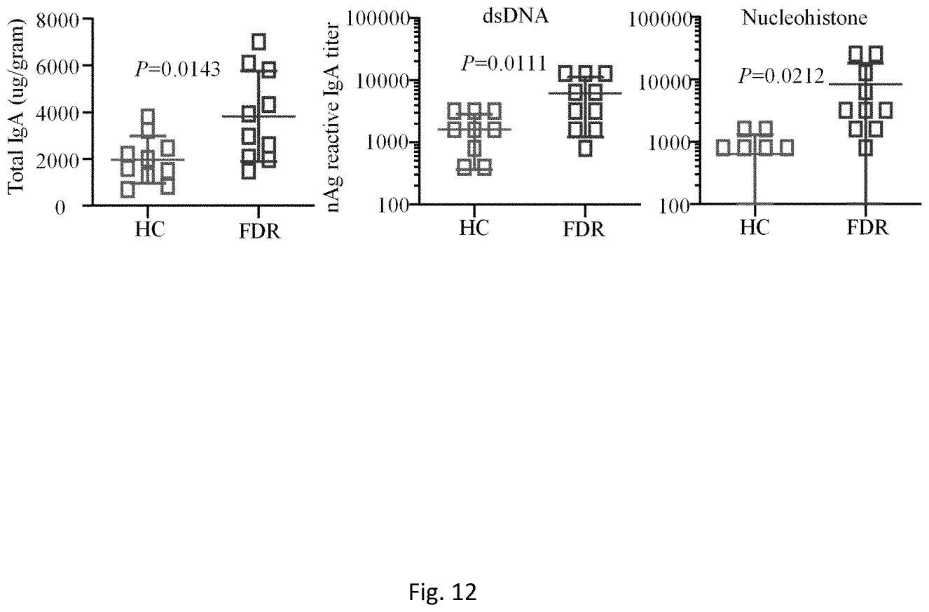

Since the ultimate goal of this research is to identify reliable evidence-based biomarkers for early detection of autoimmune progression in at-risk subjects, it is critical to determine the clinical relevance of pre-clinical observations to human SLE. With the exception of a recent report showing higher total-IgA levels in stool samples from SLE patients (Azzouz et al., 2019, Ann Rheum Dis, 78:947-956), the dynamics of fecal IgA in at-risk subjects/pre-clinical stages, and early onset SLE patients are unknown. Most importantly, nAg reactivity of fecal IgA in SLE patients or at-risk subjects have never been reported. Therefore, to realize the clinical relevance of preliminary results from a preclinical model, stool sample aliquots from small cohorts of healthy controls and SLE patients were examined for fecal IgA isotype -abundance and -nAg reactivity. FIG. 10 shows that the abundance of total-IgA and both IgA1 and IgA2 isotypes are relatively higher in SLE patients compared to healthy controls. Interestingly, dsDNA and nucleohistone ELISA revealed that fecal IgA of SLE patients possess significantly higher nAg reactivity compared to these antibodies of healthy controls (FIG. 11). These observations are of profound significance considering the fact that all SLE patients were under hydroxychloroquine treatment, which suppresses immune function and antibody production including IgA nephropathy (Bai et al., 2019, Biochem Pharmacol 169, 113619; Torigoe et al., 2018, Clin Immunol 195:1-7; James et al., 2007, Lupus 16:401-9; Yang et al., 2018, Am J Nephroi 47:145-152; Gao et al., 2017, Int Urol Nephroi 49:1233-1241). This suggests the possibility that first degree relatives (FDRs) could be predisposed to develop the disease and show profoundly higher IgA abundance and nAg reactivity. Therefore, fecal IgA levels are quantified and their nAg reactivity is determined in archived stool samples from SLE patients, antinuclear autoantibodies (ANA)-positive and ANA-negative first degree relatives (FDRs), and healthy controls (FIG. 12). These studies demonstrate that the results from the preclinical model translate to human SLE, and establish an association between fecal IgA features and systemic autoimmunity. They also demonstrate the biomarker value of fecal IgA features for predicting the disease in at-risk subjects.

### Determination of Fecal IgA Levels and nAg Reactivity

Although IgA2 is the predominantly secreted IgA in human stool, the features of both isotypes and the total-IgA are examined. Assays are performed similar to that for mouse IgA with modifications. Briefly: 1) Concentrations of IgA1, IgA2, and total-IgA are determined in optimally diluted extracts by quantitative ELISA using anti-human IgA1 and anti-human IgA2 hinge-region specific monoclonal antibody, and anti-human IgA coated ELISA plates respectively, and detected using anti-human IgA-HRP. Purified IgA, IgA1 and IgA2 (Athens Research & Technology) are used as standards. The IgA1 and IgA2 concentration/gram of stool is calculated using respective standard curves generated for each plate, and compared between groups. 2) nAg reactivity titer of stool IgA of these samples are tested using serial dilutions by ELISA using calf thymus dsDNA and nucleohistone coated plates and biotin-linked anti-human IgA1, IgA2 and IgA antibodies, and streptavidin-HRP. ELISA titer values are compared to that between groups as for IgA1, IgA2, and IgA values. To validate the nAg reactivity further, standard ANAIFA using Hep2 cell substrate slides is carried out with serial dilutions of all samples and fluorochrome linked anti-human IgA1, IgA2 and IgA antibodies. IFA titer values are compared between groups. To calculate the NRI relative to IgA1, IgA2 and IgA concentrations, dsDNA and nucleohistone reactive ELISA titers, as well as ANA IFA titers, of each sample is divided by its total IgA1, IgA2 and IgA concentration respectively. This determines the true extent of secretion of nAg reactive IgA1, IgA2 and total IgA in the feces of SLE patients and ANA+ and ANA− FDRs, compared to healthy controls.

All ELISA assays are optimized for various factors including concentration of primary and secondary reagents and antigens, sample dilutions. Known reference positive and negative controls, standards, and blank well are included in every plate where applicable to ensure the validity of the results.

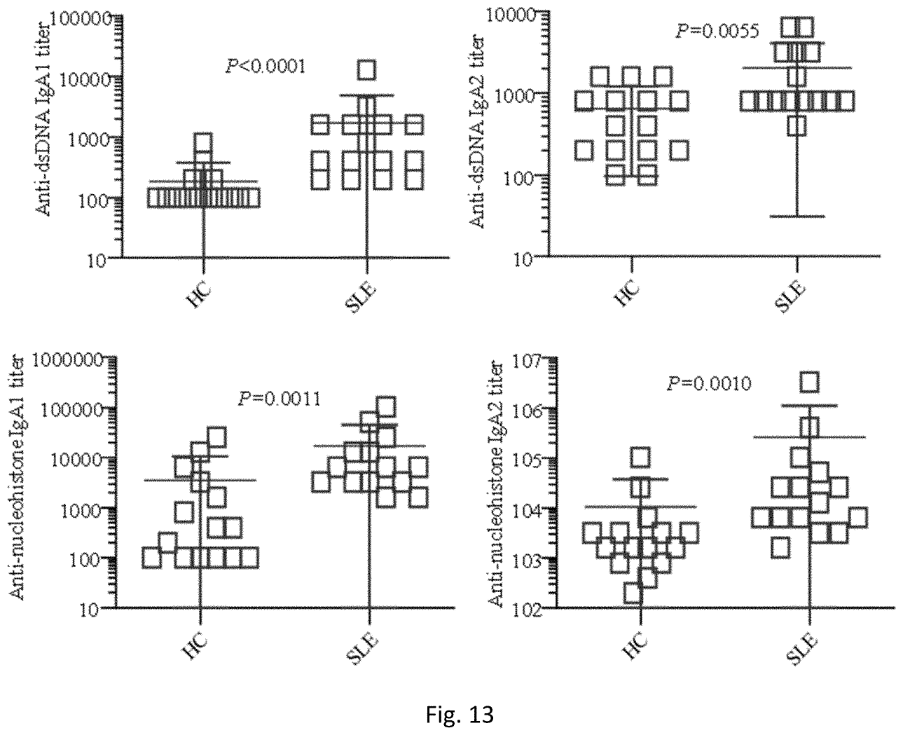

The data presented in FIG. 13 shows that, like total IgA, IgA1 and IgA2 subclasses of lupus-patients have high nuclear antigen reactivity.

### Lack of Gender Bias

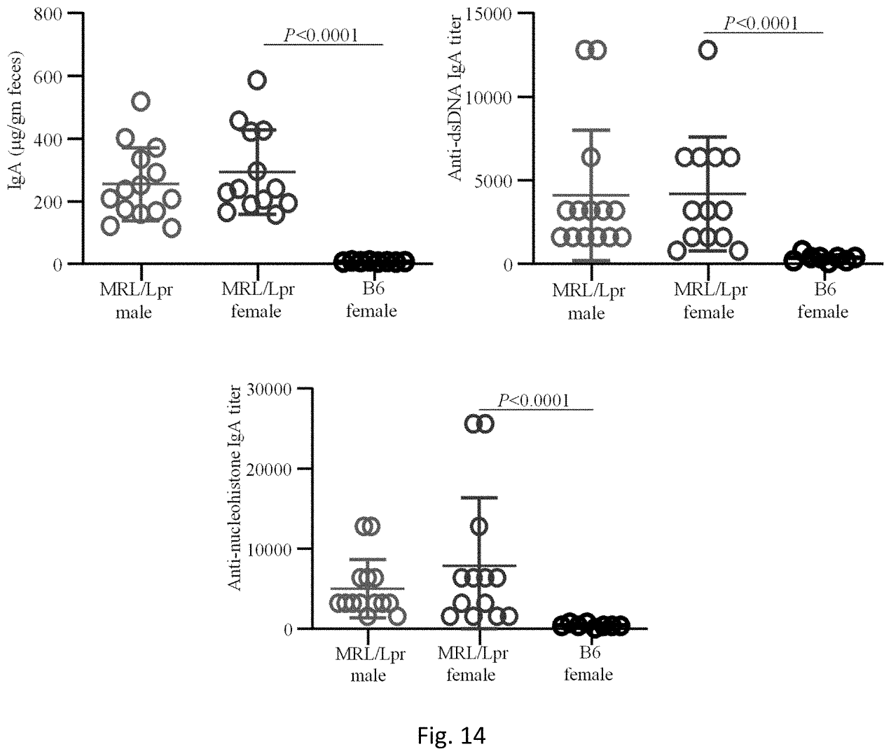

The data presented in FIG. 14 shows that there is higher abundance and nuclear antigen reactivity of fecal IgA in both male and female lupus-prone MRL/lpr mice compared to non-susceptible B6 mice. Unlike in SNF1 mice which showed gender bias, MRL/lpr mice did not show significant gender bias in fecal IgA features.

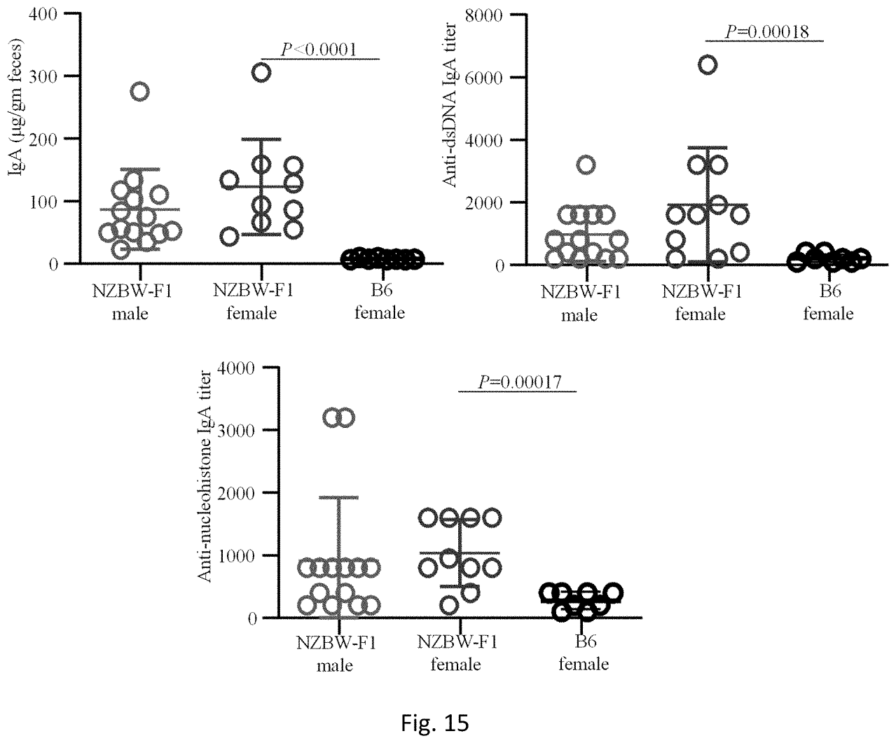

The data presented in FIG. 15 shows that there is higher abundance and nuclear antigen reactivity of fecal IgA in both male and female lupus-prone NZBW-F1 mice compared to non-susceptible B6 mice. Unlike in SNF1 mice which showed gender bias, NZBW-F1 mice did not show significant gender bias in fecal IgA features.

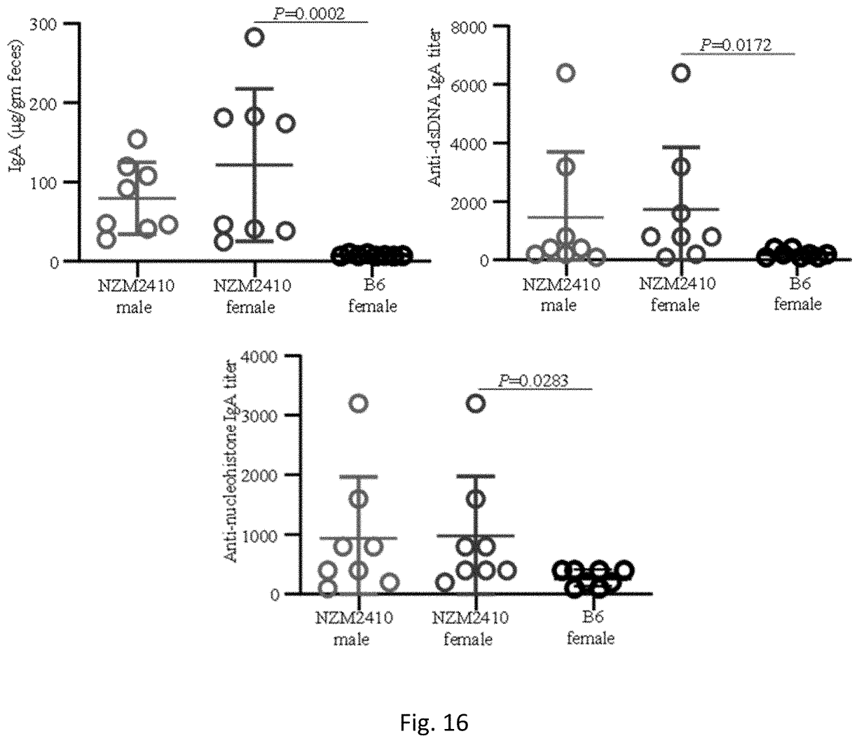

The data presented in FIG. 16 shows that there is higher abundance and nuclear antigen reactivity of fecal IgA in both male and female lupus-prone NZM2140 mice compared to non-susceptible B6 mice. Unlike in SNF1 mice which showed gender bias, NZM2140 mice did not show significant gender bias in fecal IgA features.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of detecting at least one IgA autoantibody in a subject comprising:
obtaining a fecal sample from the subject; and
contacting a portion of the sample with a capture molecule which specifically binds to at least one IgA autoantibody.

2. The method of claim 1, wherein the method detects at least one selected from the group consisting of total IgA, IgA1, IgA2 and IgA anti-nuclear antigen (IgA-ANA).

3. The method of claim 1, further comprising contacting the sample with a secondary antibody, wherein the secondary antibody is linked to a detectable moiety; and measuring a detectable signal generated from the detectable moiety.

4. The method of claim 2, wherein at least one IgA anti-nuclear antigen autoantibody is an autoantibody against a nuclear antigen selected from the group consisting of RNP/Sm, SS-A, Ro-52, SS-B, Scl-70, Pm-Scl, Jo-1, centromere B, PCNA, dsDNA, nucleosomes, nucleohistones, histones, ribozomal P-protein, and AMA-M2:

5. A method of diagnosing a subject as having or being at increased risk of an autoimmune disease, the method comprising:
a) obtaining a fecal sample from the subject;
b) contacting a portion of the sample with a capture molecule which binds to at least one IgA autoantibody;
c) contacting the sample with a detector molecule, wherein the detector molecule is linked to a detectable moiety; and
d) measuring a detectable signal generated from the detectable moiety.

6. The method of claim 5, wherein steps b-d are achieved sequentially or simultaneously in an automated fashion.

7. The method of claim 5, wherein the capture molecule comprises an anti-IgA antibody which captures the total IgA antibodies, and wherein the method further comprises a step of contacting the captured IgA total antibodies with at least one nuclear antigen prior to the step of contacting the sample with a detector molecule.

8. The method of claim 7, wherein the nuclear antigen is selected from the group consisting of: RNP/Sm, SS-A, Ro-52, SS-B, Scl-70, Pm-Scl, Jo-1, centromere B, PCNA, dsDNA, nucleosomes, nucleohistones, histones, ribozomal P-protein, and AMA-M2, or a natural or synthetic fragment thereof.

9. The method of claim 5, wherein the capture molecule comprises a natural, synthetic or recombinant nuclear antigen or a fragment thereof.

10. The method of claim 9, wherein the nuclear antigen is selected from the group consisting of: RNP/Sm, SS-A, Ro-52, SS-B, Scl-70, Pm-Scl, Jo-1, centromere B, PCNA, dsDNA, nucleosomes, nucleohistones, histones, ribozomal P-protein, and AMA-M2, or a natural or synthetic fragment thereof.

11. The method of claim 5, wherein the capture molecule is an antibody against total IgA, IgA1 or IgA2 antibody, wherein the capture molecule is used to measure the abundance of IgA antibodies.

12. The method of claim 5, wherein the detector molecule is an antibody against IgA, IgA1 or IgA2 which is linked to a detectable moiety.

13. The method of claim 5, further comprising comparing the detectable signal generated from the detectable moiety to a comparator control, and diagnosing a subject as having or being at risk of developing an autoimmune disease when the detectable signal is increased relative to the comparator control.

14. The method of claim 13, wherein the comparator control is selected from the group consisting of a negative control, an expected normal background value of the subject, a historical normal background value of the subject, an expected normal background value of a population that the subject is a member of, and a historical normal background value of a population that the subject is a member of.

15. The method of claim 5, wherein the disease or disorder is selected from the group consisting of systemic lupus erythematosus (SLE), neonatal lupus erythematosus, Sjogren's Syndrome, Sicca syndrome, Diffuse systemic sclerosis, scleroderma, rheumatoid arthritis, Juvenile idiopathic arthritis, Drug induced lupus, Polymyositis, dermatomyositis, idiopathic inflammatory myopathies (IIM), mixed connective tissue disease (MCTD), and primary biliary cholangitis (PBC).

16. The method of claim 15, wherein the subject is selected from the group consisting of a symptomatic subject, an asymptomatic subject, a subject exhibiting non-specific indicators of a disease or disorder, and a subject is receiving a cancer treatment therapy.

17. The method of claim 16, wherein the subject is receiving a cancer treatment selected from the group consisting of Ipilimumab, Durvalumab, Avelumab, Atezolizumab, Pembrolizumab, Nivolumab, and Cemiplimab.

18. The method of claim 16, further comprising administering a treatment for the diagnosed disease or disorder.

19. The method of claim 18, wherein the treatment is selected from the group consisting of immunosuppressant drugs, corticosteroids, tofacitinib, calcineurin inhibitors, antiproliferative agents, mTOR inhibitors, abatacept, adalimumab, anakinra, certolizumab, etanercept, golimumab, infliximab, ixekizumab, natalizumab, secukinumab, tacilizumab, ustekinumab, vedolizumab, basiliximab, daclizumab, muromonab, hydroxychloroquine, methotrexate, cyclosporine, lifitegrast, nonsteroidal anti-inflammatory drugs, pilocarpine, and cevimeline.

20. A method of preparing an IgA autoantibody sample comprising:
a. providing a fecal sample comprising at least one IgA autoantibody, wherein the subject is suspected of having an autoimmune disease or disorder or the subject has a symptom of an autoimmune disease or disorder;
b. selectively extracting total IgA antibodies from the fecal sample; and
c. performing an assay on the extracted total IgA antibodies in order to quantitatively or qualitatively detect the level of IgA antibodies in the sample.
